(19) United States
(12) Patent Application Publication
(10) Pub. No.: US 2026/0137406 A1
(43) Pub. Date: May 21, 2026

(54) MINIMALLY INVASIVE NASAL TURBINATE SURGERY

(71) Applicant: Spirair, Inc., South San Francisco, CA (US)

(72) Inventors: James R. KINTZING, Concord, CA (US); Brandon A. MCCUTCHEON, Orinda, CA (US); Noah R. GOLDSMITH, Santa Cruz, CA (US); Jacob A. KINTZING, Nutly, NJ (US); Andrew S. HUFFMASTER, Fremont, CA (US); Sabrina HUA, San Francisco, CA (US); Andy H. UCHIDA, Los Altos, CA (US)

(21) Appl. No.: 19/393,433

(22) Filed: Nov. 18, 2025

Related U.S. Application Data
(60) Provisional application No. 63/722,002, filed on Nov. 18, 2024.

(51) Int. Cl. A61B 17/24; A61B 17/04

(52) U.S. Cl. CPC A61B 17/24; A61B 17/0401; A61B 2017/0409; A61B 2017/0417; A61B 2017/0464

(57) ABSTRACT

Described herein are devices, systems, and methods for applying a tension force to various tissues. The devices may be delivered in a minimally invasive fashion and used to manipulate tissues in the nose (e.g., nasal septum, nasal turbinates), ear, and throat. Force may be maintained by the devices for a time period that allows shaping, compression, and/or approximation of tissues.

### CROSS-REFERENCE TO RELATED APPLICATIONS

This application claims priority to U.S. Provisional Application No. 63/722,002, filed on Nov. 18, 2024, which is hereby incorporated by reference in its entirety.

### FIELD

This application generally relates to devices, systems, and methods for applying a tension force to various tissues. The devices may be delivered in a minimally invasive manner and used to manipulate tissues in the nose, e.g., to correct nasal septal deviation or to medialize or lateralize one or more nasal turbinates. The tension force may be maintained by the devices for a time period that allows shaping, compression, repositioning, and/or approximation of tissues.

### BACKGROUND

A deviated septum may cause nasal airway obstruction, which impairs a patient's ability to breath. Oftentimes, the patient may require a septoplasty or septorhinoplasty surgery to alleviate their symptoms. Approximately 300,000-600,000 patients require these surgeries in the United States every year. While many ENT surgeries have transitioned to an office-based setting, septal surgery has lagged behind, leaving patients and physicians looking for minimally invasive approaches.

Septal surgery is non-trivial, requiring a trip to the operating room and general anesthesia. The recovery may also be significant, especially in the case of septorhinoplasty, which is performed to straighten the nasal septum and reshape the nose. For the surgeon, operating room (OR) based surgeries may present increased risks and costs while also introducing inefficiencies in the delivery of care. However, there are no clinically or cost effective minimally invasive septal correction devices that are commercially available. Accordingly, it would be useful to have new minimally invasive devices, systems, and methods for manipulating and reshaping the nasal septum. Additionally, with respect to procedures performed on the sinuses or nasal turbinates, it would be useful to have new minimally invasive devices, systems, and methods that simplify the procedures and reduce crust formation in the nasal passages.

### SUMMARY

Described herein are devices, systems, and methods for applying a tension force to various tissues. The devices may be delivered in a minimally invasive fashion and used to manipulate, e.g., reshape tissues in the nose, ear, and/or throat, as well as other tissues as described elsewhere herein. Force may be maintained for a time period that allows shaping (including reshaping), compression, repositioning, and/or approximation of tissues. The devices may include an elongate tension element having a distal anchor that may be configured for insertion in an insertion configuration into or through tissue in one direction, and upon application of force in the opposite direction, configured to swivel (e.g., turn, tilt, flip, rotate, or pivot) about a point (e.g., a pivot point) of the tension element and/or distal anchor to a deployed configuration. In the deployed configuration, the distal anchor may be prevented from passing back through the tissue. In some instances, the longitudinal axis of the distal anchor in its insertion configuration is parallel or substantially parallel to the longitudinal axis of an elongate body of the tension element and orthogonal (or non-parallel) to the longitudinal axis of the tension element elongate body in its deployed configuration. Additionally, or alternatively, the distal anchor may include an anchor body having a surface area, an insertion configuration, and a deployed configuration, where the distal anchor in the deployed configuration has a larger surface area for opposing tissue than the distal anchor in the insertion configuration. Tension may continue to be applied to the tension element and adjusted to the amount desired for the intended application. For example, tension may be adjusted to an amount that alters the shape and/or position of nasal tissues (e.g., a nasal septum, a nasal valve, a nasal turbinate). As used herein, the terms “tension element” and “shaping element” are used interchangeably throughout.

The tension element may be made from biodegradable or non-biodegradable materials. When the tension element is biodegradable, it may be made from a biodegradable polymer. Exemplary biodegradable polymers include without limitation, LPLA (Poly(L-lactide)), DLPLA (Poly(DL-lactide)), LDLPLA (Poly(DL-lactide-co-L-lactide)), LPLA-HA (Poly(L-lactide) with hydroxylapatite), PGA (Poly(glycolide)), PGA-TMC (Poly(glycolide-co-trimethylene carbonate) or polyglyconate), PDO (Poly(dioxanone)), LPLG (Poly(L-lactide-co-glycolide)), DLPLG (Poly(DL-lactide-co-glycolide), or copolymers or blends thereof. In some variations, the biodegradable polymer comprises a polylactide, a poly(orthoester), a poly(phosphoester), a polyphosphazene, a polyanhydride, a polycaprolactone, a polyurethane, a polycarbonate, chitosan, cyclodextrin, dextran, hyaluronic acid, chondroitin sulfate, dermatan sulfate, heparin, heparin sulfate, keratan sulfate, or a copolymer or blend thereof. In one variation, the tension element or a portion of the tension element may be made from PDO (Poly(dioxanone)). In another variation, the tension element may be made from a metal, for example, magnesium or a magnesium alloy. Other metals may also be used. When the tension element is non-biodegradable, it may be made from a non-biodegradable polymer.

Exemplary non-biodegradable polymers include without limitation, polypropylene, polyvinyl chloride, polyethylene, polythene terephthalate, and polystyrene. Other materials that may be used to make the tension elements include textiles and non-woven materials (e.g., materials made by electrospinning).

When the tension element is formed from a biodegradable material, it may degrade over a period of about one month, two months, three months, about four months, about five months, about six months, about seven months, about eight months, about nine months, about ten months, about eleven months, or about twelve months. In one variation, the tension element may degrade over a period ranging from about four months to about nine months. In another variation, the tension element may degrade over a period of about six months.

When the tension element is non-biodegradable, it may be made from a non-biodegradable polymer or a metal. Exemplary non-biodegradable polymers include without limitation, poly(ethylene vinyl acetate), poly(vinyl acetate), silicone polymers, polyurethanes, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chorosulphonated polyolefins, polyethylene oxide, silk, Nylon, polyamide, polypropylene, polyester, polybutester, polytetrafluoroethylene (PTFE), expanded PTFE (ePTFE), aromatic polyamides (aramids), and copolymers and blends thereof. Exemplary metals include, but are not limited to, silver, platinum, stainless steel, nickel, titanium, and alloys thereof.

The tension element may be configured to hold or maintain a force on a target tissue. The force may be a tension force ranging from about 4.0 Newtons to about 70 Newtons, including all values and sub-ranges therein, which may be generated by pulling on at least a portion of the tension element (e.g., the free proximal end of the tension element) after the distal anchor has been fixed to the target tissue. Tensile strength of the tension element may range from about 100 MPa to about 800 MPa, including all values and sub-ranges therein. In some instances, the tensile strength may be at least about 150 MPa. In other instances, the tensile strength may be at least about 300 MPa.

The length of the tension element prior to delivery may vary depending on the target tissue of deployment, type of procedure being performed, and/or the anatomy of the subject. Tension element length prior to delivery may range from about 10 cm to about 30 cm, including all values and sub-ranges therein. In one variation, the tension element may have a length of about 15 cm prior to delivery. In another variation, the tension element may have a length of about 19 cm prior to delivery. In a further variation, the tension element may have a length of about 10 cm prior to delivery. Once delivered to or through the target tissue, the tension element may be trimmed to a length that applies an appropriate amount of force, reshaping, etc., to the target tissue. The longer length may help facilitate handling of the tension element while the target tissue is being manipulated. In some variations, the elongate body of the tension element is straight. In other variations, the elongate body, or portions thereof, may have an undulating shape. In further variations, the proximal end of the elongate body may include an eyelet. The eyelet may be used to couple a suture to the tension element.

The distal anchor may be variously sized and shaped. In general, the distal anchor in its deployed configuration prevents passage of the distal end of the tension element back though tissue. In some variations, the distal anchor may include a first arm and a second arm, each having a length from a pivot point of the device. The length of the first arm from the pivot point may be different from the length of the second arm from the pivot point. In one variation, the first and second arm lengths may be the same. In another variation, the first arm length may be shorter than the second arm length. In these variations, the first, shorter arm length may range from about 1.0 mm to about 4.0 mm, including all values and sub-ranges therein, and the second, longer arm length may range from about 2.0 mm to about 8.0 mm, including all values and sub-ranges therein. For example, the shorter arm length may be about 1.0 mm, about 1.5 mm, about 2.0 mm, about 2.5 mm, about 3.0 mm, about 3.5 mm, or about 4.0 mm, and the longer arm length may be about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, or about 8.0 mm. The width of the distal anchor may range from about 0.5 mm to about 3.0 mm, including all values and sub-ranges therein. For example, the distal anchor width may be about 0.5 mm, about 0.75 mm, about 1.0 mm, about 1.25 mm, about 1.5 mm, about 1.75 mm, about 2.0 mm, about 2.25 mm, about 2.5 mm, about 2.75 mm, or about 3.0 mm. In some instances, it may be useful for the distal anchor to have a width of about 1.25 mm. The thickness of the distal anchor may be the same as the device thickness and range from about 0.25 mm to about 1.5 mm, including all values and sub-ranges therein. For example, the distal anchor thickness may be about 0.25 mm, about 0.5 mm, about 0.75 mm, about 1.0 mm, about 1.25 mm, or about 1.5 mm. In one variation, it may be useful for the distal anchor thickness to be about 0.65 mm.

Upon the application of force to the elongate body, the distal anchor may swivel (e.g., turn, tilt, flip, rotate, or pivot) about a point (e.g., a pivot point). An end of one or more of the first and second arms may have a shape configured to initiate pivoting of the distal anchor upon the application of force to the elongate body and/or facilitate its engagement against tissue. For example, the shape may include one or more of a bevel, taper, and chamfer.

A plurality of proximal anchors (e.g., migration prevention elements) may further be disposed between the distal anchor and the proximal end of the tension element. The distal anchor and the plurality of proximal anchors may be the same type of anchor or different types of anchors. In some variations, the plurality of proximal anchors may include one or more of a barb, hook, spike, nub, and protrusion. A distal end of each of the plurality of proximal anchors may be beveled, tapered, sharp, curved, or a combination thereof. The plurality of proximal anchors may be symmetrically or asymmetrically spaced apart (e.g., staggered) along a length of the elongate body. Staggering placement of the proximal anchors along the elongate body may provide a surgeon with finer tension control.

The spacing between a first proximal anchor of the plurality of proximal anchors and the distal anchor may be between about 2.0 mm to about 10 mm, including all values and sub-ranges therein. For example, the spacing between a first proximal anchor of the plurality of proximal anchors and the distal anchor may be about 2.0 mm, about 3.0 mm, about 4.0 mm, about 5.0 mm, about 6.0 mm, about 7.0 mm, about 8.0 mm, about 9.0 mm, or about 10 mm. When used to treat a nasal septal deviation, the spacing may be between about 3.0 mm to about 8.0 mm, including all values and sub-ranges therein. For example, the spacing between a first proximal anchor of the plurality of proximal anchors and the distal anchor may be about 3.0 mm, about 4.0 mm, about 5.0 mm, about 6.0 mm, about 7.0 mm, or about 8.0 mm.

When used to medialize or lateralize a nasal turbinate, the spacing may be between about 2.0 mm to about 6.0 mm, including all values and sub-ranges therein. For example, the spacing between a first proximal anchor (most distal anchor) of the plurality of proximal anchors and the distal anchor may be about 2.0 mm, about 3.0 mm, about 4.0 mm, about 5.0 mm, or about 6.0 mm. In one variation, the spacing between the distal anchor and the first proximal anchor of the plurality of proximal anchors is about 4.8 mm.

An enlarged tip may also be provided at the distal end of the tension element distal to the distal anchor to further facilitate anchoring of the tension element to tissue and/or coupling to an anchor delivery element, as further described below. The distal anchor and plurality of proximal anchors may be made from the same material as the tension element, or from different materials. In some variations, the distal anchor may be made from a non-biodegradable material, and the plurality of proximal anchors made from a biodegradable material.

The devices described herein may further include a proximal needle removably attached to the proximal end of the elongate body of the tension element. The proximal needle may be used to place or manipulate the proximal end of the elongate body through or around tissue, and may be removably attached in various ways to the tension element. For example, the proximal needle may be removable attached to the tension element by swaging or crimping, or by threading the tension element through a corresponding structure in the proximal needle.

At the distal end of the tension element, an anchor delivery element may be coupled to the distal anchor. The anchor delivery element may include an anchor support and a cutting tip configured to pass the distal anchor through the tissue in its insertion configuration. Some variations of the anchor delivery element may include an anchor support having a seating region configured to removably secure the anchor to the anchor delivery element. The seating region may be shaped to correspond to the shape of the distal anchor. Furthermore, the seating region may include an area of decreased thickness in order to level the surface of the distal anchor with the anchor support when the distal anchor is seated on the anchor delivery element. Leveling in this manner may present a flush surface to tissue that may prevent the distal anchor and/or anchor delivery element from catching on tissue during insertion. However, in other variations, the anchor support may not include an area of decreased thickness. In some instances, the seating region may have a size and shape that allows the distal anchor to couple to the seating region via an interference fit. For example, the distal anchor and seating region may have the same shape, but the distal anchor may be sized slightly larger than the seating region such that it may be press fit therein. In other variations, the anchor support may not include an area of decreased thickness.

The cutting tip of the anchor support may have varying amounts of curvature that may help a user more easily track the cutting tip against tissue and initiate a puncture within the tissue (providing a tactile advantage to the user). In these variations, the vertex of the angle of curvature may be located at the distal end of the distal tip or at the proximal end of the distal tip. When the vertex is located at the distal end of the distal tip, the angle of curvature may range from about 15 degrees to about 45 degrees, including all values and sub-ranges therein. For example, the angle of curvature may be about 15 degrees, about 20 degrees, about 25 degrees, about 30 degrees, about 35 degrees, about 40 degrees, or about 45 degrees. In some instances, it may be useful for the angle of curvature to be about 20 degrees. In other instances, it may be useful for the angle of curvature to range from about 10 degrees to about 30 degrees. When the vertex is located at the proximal end of the distal tip, the angle of curvature may range from about 10 degrees to about 45 degrees, including all values and sub-ranges therein. For example, the angle of curvature may be about 10 degrees, about 15 degrees, about 20 degrees, about 25 degrees, about 30 degrees, about 35 degrees, about 40 degrees, or about 45 degrees. The distal cutting tip may also have a sharpness and/or shape that allows the user to more easily track the distal tip against tissue and initiate a puncture within the tissue (providing a tactile advantage to the user).

The tissues that may be reshaped with the devices describe herein include without limitation, nasal tissues, throat tissues, and ear tissues. Non-limiting examples of nasal tissue include nasal septal cartilage, lateral nasal cartilage, major alar cartilage, minor alar cartilage, alar fibrofatty tissue, nasal bone, or a nasal turbinate. Exemplary throat tissues include without limitation, the uvula, soft palate, laryngeal cartilage, thyroid cartilage, cricoid cartilage, epiglottis, and tonsils. Non-limiting examples of ear tissues include cartilage of the helix, anti-helix, tragus, anti-tragus, superior crus, fossa triangularis, concha, and connective tissue of the earlobe. The devices may also be used to manipulate or shape cartilage, bone, or other tissues in orthopedic applications, or to manipulate or shape vascular, heart, or other tissues in cardiovascular applications. The devices may further be used in cosmetic applications to reshape or support tissue. The devices may also be used in urologic or gynecologic applications to reshape or support tissue. For example, the devices may be used to alter the shape of penile curvature. The device may also be used to support pelvic floor muscles. In some instances, the devices described herein may be used to splint, hold, or support a tissue.

In addition to a distal anchor and a plurality of proximal anchors, some of the tension elements described herein may include a retainer on the elongate body configured to releasably attach to a portion of a delivery system used to implant the tension element into tissue. The retainer may be disposed on the elongate body between the distal anchor and a first proximal anchor of the plurality of proximal anchors. As further described below, in some variations, the retainer may be used to releasably attach the tension element to a pusher of the delivery system via a coupler of the pusher. In these variations, the distal anchor may include a first arm and a second arm, where the first and second arms have a different length when measured from a pivot point of the tension element. The aforementioned tension element may be useful when reshaping nasal septal tissue, e.g., to treat nasal septal deviation.

When manipulation of one or more nasal turbinates is desired, the tension element may include an elongate body having a distal and a proximal end, a distal anchor at the distal end comprising at least a first arm and a second arm (e.g., a T-anchor), and a plurality of proximal anchors disposed along a region/length of the elongate body between the distal anchor and the proximal end, where the size of the plurality of proximal anchors decreases along the region/length of the elongate body from the distal anchor towards the proximal end. Stated differently, the tension element may include a plurality of proximal anchors having a decreasing size gradient along a region/length of the elongate body from the distal anchor towards the proximal end. The distance (e.g., spacer length) between the distal anchor and the first proximal anchor of the plurality of proximal anchors may range from about 4.0 mm to about 6.0 mm, including all values and sub-ranges therein. As further described below, when middle turbinates are to be medialized towards the nasal septum, the spacer length may be selected such that none of the plurality of proximal anchors contacts the nasal septum after tensioning and securement of the elongate body. The plurality of proximal anchors may be staggered along a length of the elongate body. Additionally, the region/length of the elongate body including the proximal anchors may be configured to be undulating. The region/length may be a predetermined length ranging from about 10 mm to about 15 mm, including all values and sub-ranges therein.

Delivery systems for reshaping tissues are also described herein. These systems may generally include a pusher and a device including a tension element. The pusher may include a proximal end, a distal end, a length therebetween, and a coupler (connector) at the distal end. The tension element may include an elongate body, a pivot point, and a distal anchor configured to releasably attach to the coupler of the pusher. In some variations, the coupler is configured to releasably attach to a retainer of tension element, as further described below. As previously mentioned, the distal anchor may include an anchor body and/or first and second arms having a length. The length of the first arm from a pivot point of the device may be different from the length of the second arm from the pivot point. For example, the first arm length may be shorter than the second arm length. The device may include a plurality of proximal anchors disposed between the distal anchor and the proximal end of the elongate body of the tension element, as also previously mentioned.

When the tension elements include a retainer, the delivery systems may include a handle and a cannula extending from the handle. The cannula may include a proximal end and a distal portion, where the distal portion may comprise a curved upper surface, a sharp distal tip, and a lower surface comprising a first cutting edge and a second cutting edge, where the first and second cutting edges and the sharp distal tip are configured to create a U-shaped incision (slit) in a target nasal tissue. For example, the incision may create a shaped path, e.g., an arc following a smooth path like a letter ‘U’. In other instances, the incision may create an angular path like a letter ‘V’ or part of a rectangle ‘|_|’, or a combination of a smooth and angular paths. After deployment from the delivery device, the elongate body of the tension element may be seated in the incision, and the distal anchor implanted in tissue behind the tissue flap created by the U-shaped incision. In contrast to the puncture holes made by cannulas of conventional delivery systems, the U-shaped incision and/or tissue flap may help prevent dislodgement of the distal anchor when a force is applied to the tension element. This improved delivery system may also include a pusher disposed within the cannula, where the pusher may comprise a proximal end, a distal end, and a coupler at the distal end configured for releasable attachment to the retainer of the reshaping device.

The pusher of the system may be flexible, and may include a plurality of slits along its length. For example, the pusher may comprise a hypotube including a plurality of laser cuttings (laser cut hypotube). The plurality of slits/cuts may be symmetrically or asymmetrically spaced apart along the length of the pusher. The plurality of slits/cuts may be identically sized or may change in size along the length of the pusher or on different sides of the pusher. Some variations of the system may further include a cannula with a distal end having an angle of curvature configured to decrease the amount of force applied to advance a distal tip of the cannula through tissue (providing a mechanical advantage to the user). The plurality of slits/cuts along the length of the pusher may assist with advancement of the pusher through the curve of the cannula.

In some variations, the cannula of the delivery device may be made from a polymer. Exemplary polymers include, but are not limited to, acrylic, polycarbonate, polyethylene terephthalate, polyvinyl chloride, polyethylene, polypropylene, and polystyrene. Transparent forms of the aforementioned polymers may be used to make a transparent cannula. In other variations, the cannula may be made from stainless steel or other suitable metals. The cannula may include markings thereon to help facilitate tension element placement. When made from a metal, the markings may be laser markings.

The cannula may also have various cross-sectional shapes. For example, the cross-sectional shape of the cannula may be circular, non-circular, semi-circular, or ovular. In some variations where the cannula cross-section is noncircular, the shape may facilitate orientation of the cannula. One or more ports in fluid communication with the lumen may be provided in the cannula for delivery of the tensioning element from the lumen into tissue. The one or more ports may be provided in any suitable location on the cannula, for example, at the distal tip of the cannula or distal side wall of the cannula. The one or more ports may also have any suitable shape. For example, the one or more ports may be circular, semi-circular, or ovular. When a port is provided at the distal tip of the cannula, the port may have a length and a depth. The side profile of the port may also include a curved portion and a flat portion.

Further variations of the delivery system may include a lumen or hood coupled to the cannula and configured to apply suction to tissue to help with deployment of the cannula distal tip through the tissue. The hood may be configured to protect tissue from damage by the sharp cannula distal tip, e.g., while being advanced through the mucosal tunnel, as well as limit the depth of advancement of the cannula distal tip into tissue.

Methods for reshaping a tissue in a subject are also described herein. The methods may generally include securing a tension element to the tissue, where the tension element comprises an elongate body having a proximal end and a distal end, a pivot point, and a distal anchor at the tension element distal end having a first arm and a second arm. The length of the first arm from the pivot point may be different from the length of the second arm from the pivot point.

In some variations, the distal end of the tension element may be directed through the tissue with an anchor delivery element. The distal anchor may include an anchor body that is configured to swivel (e.g., turn, tilt, flip, rotate, or pivot) about the pivot point. The distal anchor may also have an insertion configuration and a deployed configuration as described above and further herein. After securing the tension element to tissue, a force may be applied to the elongate body to swivel the distal anchor at or about the point from the insertion configuration to the deployed configuration. The force appropriate to manipulate the tissue may then be adjusted by adjusting the tension of the tension element. In some variations, the tension element may also include a plurality of proximal anchors disposed between the distal anchor and the proximal end of the tension element.

In one variation, the method for reshaping a nasal tissue of a subject may include advancing a cannula toward the nasal tissue, and advancing a device including a tension element having an elongate body, a pivot point, and a distal anchor through the cannula. The distal anchor may include a first arm and a second arm, and the first arm length from the pivot point may be different than the second arm length from the pivot point. Additionally, the method may include advancing the distal anchor in an insertion configuration from the cannula through the nasal tissue, and applying a force to the elongate tension element to rotate the distal anchor about a pivot point to transition the distal anchor to a deployed configuration. The device may or may not be preloaded within the cannula. The method may also include advancing one or more proximal anchors through nasal tissue. In some variations, the distal anchor may be advanced using a pusher.

In other variations, the device may be delivered by inserting a cannula through an access site in submucosal tissue on a first side of the nasal septum and through the nasal septum to a second side of the nasal septum. A distal anchor of a tension element may then be secured into nasal cartilage on the second side of the nasal septum, where the distal anchor comprises a pivot point and has an insertion configuration and a deployed configuration. The method may include securing the distal anchor by applying a force to the elongate tension element to swivel (e.g., turn, tilt, flip, rotate, or pivot) the distal anchor at the pivot point from the insertion configuration to the deployed configuration. In some variations, the method may further include passing a proximal end of the elongate tension element back through the access site in submucosal tissue to the second side of the nasal septum, tensioning the elongate tension element to a tensioned state, and securing the proximal end of the elongate tension element in its tensioned state to tissue on the second side of the nasal septum. In further variations, the proximal end of the elongate tension element may be passed back through the access site in submucosal tissue multiple times similar to a running suture technique to increase the retention force and/or provide appropriate correction to a deviation. The tension element in its tensioned state may apply a force ranging from about 1.0 Newton to about 70 Newtons (including all values and sub-ranges therein) to the one or more nasal tissues.

In a further variation, the method for reshaping a nasal tissue of a subject may include creating a U-shaped incision in nasal tissue with a cannula, advancing a device through the cannula, the device comprising a tension element having an elongate body, a distal anchor, and a pivot point, where the distal anchor comprises a first arm and a second arm, and wherein the length of the first arm from the pivot point is different from the length of the second arm from the pivot point; advancing the distal anchor in an insertion configuration from the cannula through the nasal tissue; and applying a force to the tension element to rotate the distal anchor about the pivot point and transition the distal anchor to the deployed configuration.

When the procedure involves manipulation of nasal turbinates, the method may include advancing a tension element through a first nasal turbinate on a first side of a nasal septum, the tension element comprising an elongate body having a proximal end and a distal end, a distal anchor at the distal end comprising at least a first arm and a second arm, and a plurality of proximal anchors disposed along a length of the elongate body between the distal anchor and the proximal end, where a size of the plurality of proximal anchors decreases along the length from the distal anchor towards the proximal end; advancing the tension element through the nasal septum; advancing the tension element through a second nasal turbinate on a second side of the nasal septum opposite the first side; and applying a force to the tension element to position the distal anchor against an outer surface of the first nasal turbinate and one or more of the plurality of proximal anchors within the second turbinate. The method may be particularly useful in medializing the middle turbinates, but may be used to manipulate, shape/reshape, position/reposition other types of nasal turbinates.

The proximal and distal ends of the elongate body of the tension element may be secured to the same tissue. Alternatively, the proximal and distal ends of the elongate body may be secured to different tissues. The tissue may be a nasal tissue, a throat tissue, or an ear tissue. Exemplary nasal tissues include without limitation, nasal septal cartilage, lateral nasal cartilage, major alar cartilage, minor alar cartilage, alar fibrofatty tissue, nasal bone, or a nasal turbinate. Exemplary throat tissues include without limitation, the uvula, soft palate, laryngeal cartilage, thyroid cartilage, cricoid cartilage, epiglottis, and tonsils. Non-limiting examples of ear tissues include cartilage of the helix, anti-helix, tragus, anti-tragus, superior crus, fossa triangularis, concha, and connective tissue of the earlobe.

The methods described herein may be used to treat various conditions and manipulate various tissues. For example, the manipulation of tissue by the tension elements may be used to treat nasal septal deviation, lateral nasal valve collapse, and other causes of nasal airway obstruction. Additionally, the manipulation of tissue may be used to medialize a middle turbinate, compresses or lateralize the inferior turbinate, or reapproximate nasal mucosa. Furthermore, the manipulation of tissue by the tension elements may alter the shape of various tissues. For example, the shape of a nasal tissue, a throat tissue, or an ear tissue may be altered.

The force applied to manipulate or shape a tissue may range from about 4.0 Newtons to about 70 Newtons. The applied force may decrease over time as the tension element biodegrades. In general, the tension element biodegrades over a period of about three months to about twelve months. For example, the tension element may biodegrade over a period of at least about four months, over a period of at least about six months, or over a period of at least about nine months.

### BRIEF DESCRIPTION OF THE DRAWINGS

FIG. 1 depicts a top view of an exemplary tension element including a distal anchor having arms that swivel from an insertion configuration to a deployed configuration.

FIG. 2 depicts an enlarged view of the distal end of the tension element shown in FIG. 1.

FIG. 3A depicts a top view of exemplary Z-Flex anchor including a centrally positioned pivot point.

FIG. 3B depicts a top view of an exemplary Y-Flex anchor including a centrally positioned pivot point.

FIGS. 4A-4C depict other exemplary distal anchors.

FIGS. 5A-5C depict yet further exemplary distal anchors.

FIGS. 6A-6F depict exemplary proximal anchors.

FIGS. 7A-7C depict exemplary eyelet shapes at the proximal end of tension elements.

FIG. 7D depicts a suture coupled to the eyelet shown in FIG. 7C.

FIGS. 8A-8C depict an exemplary anchor delivery element.

FIG. 9 depicts an exemplary Z-Flex anchor seated within the anchor delivery element of FIGS. 8A-8C.

FIG. 10 depicts an exemplary distal anchor surface being level with the anchor delivery element surface due to a decreased anchor support thickness.

FIG. 11 depicts an exemplary full thickness anchor support.

FIG. 12 depicts an exemplary cutting tip component having a tip bias.

FIG. 13A depicts another exemplary cutting tip component having a cutting profile. Use of the cutting tip component may result in the tissue defect shown in FIG. 13B.

FIG. 14A depicts another exemplary cutting tip component having a tapered profile. Use of the tapered tip component may result in the tissue defect shown in FIG. 14B.

FIGS. 15A-15G depict other exemplary cutting tips having varying amounts of beveling, taper, and/or curvature.

FIGS. 16A-16C illustrate how a curved cutting tip may improve the angle of entry into tissue.

FIGS. 17A-17C depict exemplary delivery devices including rack and pinion mechanisms for deployment of an anchor delivery element.

FIGS. 18A-18E depict another exemplary anchor delivery element including a cutting tip and a pusher.

FIG. 19A depicts an exemplary cannula angle for deployment of a distal anchor into tissue and FIG. 19B depicts laser markings on an exemplary cannula.

FIGS. 20A-20D depict exemplary distal end configurations for a cannula.

FIGS. 21A-21D illustrate an exemplary method for advancing a tension element through tissue and transitioning the distal anchor from an insertion configuration to a deployed configuration.

FIGS. 22A-22D depict an exemplary suction hood.

FIG. 23 depicts an exemplary tension element for use in nasal turbinate procedures.

FIGS. 24A-24C depict exemplary methods for medializing the middle turbinate.

FIGS. 25A-25D depict exemplary methods for treating inferior turbinate hypertrophy.

FIGS. 26A-26C depict another exemplary method for medializing middle turbinates.

FIGS. 27A-27D depict exemplary methods for treating lateral nasal valve collapse.

FIGS. 28A-28C depict an exemplary method for nasal tip reshaping.

FIG. 29 depicts an exemplary method for tacking nasal mucosa to prevent nasal septal hematoma formation.

FIGS. 30A-30C depict an exemplary method of elevating the uvula and soft palate to treat obstructive sleep apnea.

FIGS. 31A and 31B depict exemplary areas of the ear for placement of a tension element to reshape the ear.

FIGS. 32A-32E depict an exemplary method of shaping a nasal septum using a tension element.

FIG. 33 depicts another exemplary method for placing a tension element to reshape a deviated nasal septum.

FIGS. 34A-34D depict yet further exemplary distal anchor configurations.

FIG. 35 depicts a side view of an exemplary tension element including a pivot point, a retainer, and a distal anchor including a first arm and second arm, the first arm having a different length from the pivot point than the second arm.

FIG. 36 depicts a side view of another exemplary tension element for use in nasal turbinate procedures.

FIGS. 37A and 37B show an exemplary arc-shaped incision. In FIG. 37A, the incision is compared to a puncture hole made in nasal tissue; and in FIG. 37B, an exemplary distal anchor is shown lodged behind the incision.

FIGS. 38A-38G depict an exemplary delivery system configured to create an arc-shaped incision. FIGS. 38A-38C show the delivery system without a tip protector and depth guard; FIG. 38D shows the delivery system including both the tip protector and the depth guard; and FIGS. 38E-38G show the delivery system with the depth guard attached.

FIGS. 39A-39C depict enlarged views of the curved distal end of the cannula of the exemplary delivery system shown in FIGS. 38A-38G. FIG. 39A shows a perspective view of the curved distal end; FIG. 39B shows a top view of the curved distal end; and FIG. 39C shows the underside of the curved distal end.

FIGS. 40A and 40B depict the cannula of the delivery system shown in FIGS. 38A-38G with an exemplary pusher therein.

### DETAILED DESCRIPTION

Described herein are devices, systems, and methods for applying a tension force to various tissues. The devices may be delivered in a minimally invasive fashion and used to manipulate tissues in the nose, ear, and throat. The tension force may be maintained for a time period that allows shaping, compression, or approximation of tissues. The devices may include a tension element having a distal anchor that may be advanced in an insertion configuration into or through tissue in one direction, and upon application of force in the opposite direction, may swivel, tilt, turn, pivot, flip, or rotate to a deployed configuration to prevent passage of the distal anchor back through the tissue. For example, in the deployed configuration, the distal anchor may have a larger surface for tissue contact and profile that prevents passage of the distal anchor back through the tissue. Once the distal anchor has transitioned to the deployed configuration, additional force may be applied to the tension element and adjusted to the amount desired for the intended application. For example, the tension element may be placed in one or more nasal tissues and the tension adjusted to an amount that alters the shape of nasal tissues. Conditions such as nasal septal deviation, lateral nasal valve collapse, and other causes of nasal airway obstruction may be treated in this manner.

### Devices

### Tension Elements

The devices for reshaping a tissue in a subject generally include a tension element, where the tension element includes an elongate body having a proximal end and a distal end. A distal anchor having an insertion configuration and a deployed configuration may be provided at the tension element distal end and include an anchor body and a pivot point. Upon application of force to the elongate body, the distal anchor may be configured to swivel, tilt, turn, rotate, or pivot about a point (e.g., a pivot point) to transform from the insertion configuration to the deployed configuration, as stated above. In the deployed configuration, the distal anchor generally fixes or anchors the distal end of the tension element within tissue. For example, when a force is applied to the tension element proximal end, the distal anchor may swivel to flatten (and increase its contact surface area) against tissue (e.g., nasal cartilage), to prevent removal of the tension element therefrom. This applied force may generally be in the direction opposite to the direction of insertion. In some instances, the longitudinal axis of the distal anchor in its deployed configuration may be orthogonal to the longitudinal axis of the tension element. The tension element may also include a plurality of proximal anchors between the distal anchor and the proximal end of the elongate body. A needle may further be provided at the proximal end of the elongate body to facilitate advancement or placement of the tension element through tissue after the distal anchor is deployed.

The tension element may be made from biodegradable or non-biodegradable materials. When the tension element is biodegradable, it may be made from a biodegradable polymer. Exemplary biodegradable polymers include without limitation, LPLA (Poly(L-lactide)), DLPLA (Poly(DL-lactide)), LDLPLA (Poly(DL-lactide-co-L-lactide)), LPLA-HA (Poly(L-lactide) with hydroxylapatite), PGA (Poly(glycolide)), PGA-TMC (Poly(glycolide-co-trimethylene carbonate) or polyglyconate), PDO (Poly(dioxanone)), LPLG (Poly(L-lactide-co-glycolide)), DLPLG (Poly(DL-lactide-co-glycolide), or copolymers or blends thereof. In some variations, the biodegradable polymer comprises a polylactide, a poly(orthoester), a poly(phosphoester), a polyphosphazene, a polyanhydride, a polycaprolactone, a polyurethane, a polycarbonate, chitosan, cyclodextrin, dextran, hyaluronic acid, chondroitin sulfate, dermatan sulfate, heparin, heparin sulfate, keratan sulfate, or a copolymer or blend thereof. In one variation, the tension element may be made from PDO (Poly(dioxanone)). In another variation, the tension element may be made from a metal, for example, magnesium or a magnesium alloy. Other metals may also be used.

The ultimate tensile strength of polymer layers having a high ultimate tensile strength may be greater than about 150 MPa. In some variations, high ultimate tensile strengths may range from about 175 MPa to about 400 MPa, including all values and sub-ranges therein. For example, high ultimate tensile strengths may be 175 MPa, about 200 MPa, about 225 MPa, about 250 MPa, about 275 MPa, about 300 MPa, about 325 MPa, about 350 MPa, about 375 MPa, or about 400 MPa. Exemplary therapeutic agents that may be contained in a polymer layer include without limitation, an antibacterial agent, an anti-inflammatory agent, a growth promoting agent, a hemostatic agent, a clot prevention agent, an analgesic, and combinations thereof. Exemplary antibacterial agents include without limitation, aminoglycosides, amphenicols, ansamycins, bacitracin, betalactams, β-lactams such as penicillins, lincosamides, macrolides, nitrofurans, quinolones, sulfonamides, sulfones, tetracyclines, vancomycin, and any of their derivatives, and combinations thereof. Examples of penicillins that can be suitable for use with the described devices and methods include, but are not limited to, amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, cefazolin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, and ticarcillin, and combinations thereof. Cephalosporins such as cefazolin or cephalexin may also be used. The anti-inflammatory agents that may be used with the described devices and methods include without limitation, steroids such as dexamethasone and hydrocortisone. Exemplary growth factors include without limitation, TGF-β (transforming growth factor-β), BMP-2 (bone morphogenetic protein-2), BMP-7 (bone morphogenetic protein-7), BMP-12 (bone morphogenetic protein-12), BMP-13 (bone morphogenetic protein-13), IGF-I (insulin growth factor-I), FGF-2 (fibroblast growth factor-2), FGF-4 (fibroblast growth factor-14), FGF-8 (fibroblast growth factor-8), FGF-18 (fibroblast growth factor-18), and PDGF (platelet-derived growth factor), VEGF (vascular endothelial growth factor), Wnt3a, Wnt7a, and combinations thereof.

When the tension element is formed from a biodegradable material, it may degrade over a period ranging from about three months to about twelve months. For example, the tension element may degrade over a period of about one month, about two months, three months, about four months, about five months, about six months, about seven months, about eight months, about nine months, about ten months, about eleven months, or about twelve months. In one variation, the tension element may degrade over a period ranging from about four months to about nine months. Depending on the material the tension element is made from, the loss of tensile strength may occur before complete degradation of the tensile element. In these variations, the tension element may be made from a material providing a sufficient amount of tensile strength over the desired time period.

When the tension element is non-biodegradable, it may be made from a non-biodegradable polymer or a metal. Exemplary non-biodegradable polymers include without limitation, poly(ethylene vinyl acetate), poly(vinyl acetate), silicone polymers, polyurethanes, polysaccharides such as a cellulosic polymers and cellulose derivatives, acyl substituted cellulose acetates and derivatives thereof, copolymers of poly(ethylene glycol) and poly(butylene terephthalate), polystyrenes, polyvinyl chloride, polyvinyl fluoride, poly(vinyl imidazole), chorosulphonated polyolefins, polyethylene oxide, silk, Nylon, polyamide, polypropylene, polyester, polybutester, and copolymers and blends thereof. Exemplary metals include, but are not limited to, platinum, silver, stainless steel, nickel, titanium, and alloys thereof.

The tension element may be formed to have any suitable cross-sectional shape. For example, the cross-sectional shape (without any anchors included) may be circular, semi-circular, ovular, rectangular, square, or triangular. The width and/or thickness of the tension element may range from about 0.25 mm to about 1.5 mm, including all values and sub-ranges therein. For example, the width and/or thickness may be about 0.25 mm, about 0.3 mm, about 0.4 mm, about 0.5 mm, about 0.6 mm, about 0.65 mm, about 0.7 mm, about 0.75 mm, about 0.8 mm, about 0.85 mm, about 0.9 mm, about 0.95 mm, about 1.0 mm, about 1.25 mm, or about 1.5 mm. In one variation, the width and/or thickness of the tension element may be about 0.65 mm. In another variation, the width and/or thickness of the tension element may be about 0.7 mm. The tension element may have various types of elongate body configurations. For example, the elongate body may be straight, undulating, wave-like, oscillating, or include any number of pre-formed curves. An oscillating or undulating elongate body may allow for some stretch and/or increased flexibility of the tension element during tensioning.

The tension element including, for example, a distal anchor, proximal anchors, distal toe, and/or any other tension element component may be die cut from one or more polymer materials, as described above. In other variations, the tension element or any component thereof, may be formed by injection molding, three-dimensional (3D) printing, or other additive manufacturing techniques (e.g., other techniques that use computer-aided design software or 3D object scanners).

The tension element may be configured to hold or maintain a force on a target tissue. The force may be a tension force ranging from about 4.0 Newtons to about 70 Newtons, including all values and sub-ranges therein, which may be generated by pulling on the free proximal end of the tension element after the distal anchor has been fixed to the target tissue. For example, the tension force may be about 4.0 Newtons, about 5.0 Newtons, about 10 Newtons, about 15 Newtons, about 20 Newtons, about 25 Newtons, about 30 Newtons, about 35 Newtons, about 40 Newtons, about 45 Newtons, about 50 Newtons, about 55 Newtons, about 60 Newtons, about 65 Newtons, or about 70 Newtons. Tensile strength of the tension element may range from about 100 MPa to about 800 Mpa, including all values and sub-ranges therein. For example, the tensile strength may be about 100 Mpa, about 110 Mpa, about 120 Mpa, about 130 Mpa, about 140 Mpa, about 150 Mpa, about 155 Mpa, about 160 Mpa, about 165 Mpa, about 170 Mpa, about 175 Mpa, about 180 Mpa, about 185 Mpa, about 190 Mpa, about 195 Mpa, about 200 Mpa, about 210 Mpa, about 220 Mpa, about 230 Mpa, about 240 Mpa, about 250 Mpa, about 260 Mpa, about 270 Mpa, about 280 Mpa, about 290 Mpa, about 300 Mpa, about 350 Mpa, about 400 Mpa, about 450 Mpa, about 500 Mpa, about 550 Mpa, about 600 Mpa, about 650 Mpa, about 700 mPa, about 750 Mpa, or about 800 Mpa. In some instances, the tensile strength of the tension element may be at least about 150 Mpa. In other instances, the tensile strength of the tension element may be at least about 300 Mpa.

The total length of the tension element prior to delivery may vary depending on the target tissue of deployment, type of procedure being performed, and/or the anatomy of the subject. Total tension element length prior to delivery may range from about 10 cm to about 30 cm, including all values and sub-ranges therein. For example, the total length may be about 10 cm, about 11 cm, about 12 cm, about 13 cm, about 14 cm, about 15 cm, about 16 cm, about 17 cm, about 18 cm, about 19 cm, about 20 cm, about 21 cm, about 22 cm, about 23 cm, about 24 cm, about 25 cm, about 26 cm, about 27 cm, about 28 cm, about 29 cm, or about 30 cm. In one variation, the tension element may have a total length of about 15 cm prior to delivery. Once delivered to the target tissue, the tension element may be trimmed to a length that applies an appropriate amount of force, reshaping, etc., to the target tissue. The longer length may help facilitate handling of the tension element while the target tissue is being manipulated.

The length of the tension element between the distal anchor and a most distally positioned proximal anchor (e.g., a first proximal anchor) may range from about 2.0 mm to about 25 mm, including all values and sub-ranges therein. For example, this length may be about 2.0 mm, about 3.0 mm, about 4.0 mm, about 5.0 mm, about 6.0 mm, about 7.0 mm, about 8.0 mm, about 9.0 mm, about 10 mm, about 11 mm, about 12 mm, about 13 mm, about 14 mm, about 15 mm, about 16 mm, about 17 mm, about 18 mm, about 19 mm, about 20 mm, about 21 mm, about 22 mm, about 23 mm, about 24 mm, or about 25 mm. The length may be adjusted to be longer or shorter depending on the tissue to be reshaped, repositioned, and/or manipulated. Lengths between about 10 mm to about 25 mm may be useful when the nasal septal cartilage is to be manipulated. A length between about 4.0 mm to about 5.0 mm between the distal anchor and a most distally positioned proximal anchor (e.g., a first proximal anchor) may be useful when one or more turbinates is to be reshaped, repositioned, and/or manipulated.

The length of the tension element between the needle at the proximal end of elongate body and the most proximally positioned proximal anchor ranges from about 50 mm to about 70 mm, including all values and sub-ranges therein. For example, this length may be about 50 mm, about 55 mm, about 60 mm, about 65 mm, or about 70 mm. The tension element may or may not include any proximal anchors along this length. Additionally, the tension element may be trimmed to a final length along this length.

### Anchors

The devices described herein may include a distal anchor at the distal end of the tension element. The distal anchor may have an insertion configuration and a deployed configuration. Additionally, the distal anchor may include an anchor body and a pivot point. Upon application of force to the elongate body of the tension element, the distal anchor may be configured to swivel, tilt, turn, flip, rotate, or pivot about the pivot point to transform from the insertion configuration to the deployed configuration. This applied force is generally in the direction opposite to the direction of insertion. In some instances, the longitudinal axis of the distal anchor in its deployed configuration may be orthogonal to the longitudinal axis of the tension element. However, the distal anchor may swivel about the pivot point in any suitable amount to achieve a deployed configuration. The distal anchor may swivel about the pivot point at a swivel angle ranging from about 30 degrees to about 90 degrees with respect to the longitudinal axis of the tension element, including all values and sub-ranges therein. For example, the swivel angle may be about 30 degrees, about 35 degrees, about 40 degrees, about 45 degrees, about 50 degrees, about 55 degrees, about 60 degrees, about 65 degrees, about 70 degrees, about 75 degrees, about 80 degrees, about 85 degrees, or about 90 degrees.

The distal anchor may be variously sized and shaped. For example, the distal anchor may be T-shaped or X-shaped. In general, the distal anchor in its deployed configuration may prevent passage of the distal end of the tension element back though tissue. In some variations, the distal anchor may have a rounded cone shape, and a length ranging from about 0.5 mm to about 15 mm, including all values and sub-ranges therein, and a width ranging from about 0.5 mm to about 5.0 mm, including all values and sub-ranges therein. For example, the distal anchor length may be about 0.5 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.5 mm, about 9.0 mm, about 9.5 mm, about 10 mm, about 10.5 mm, about 11.0 mm, about 11.5 mm, about 12.0 mm, about 12.5 mm, about 13.0 mm, about 13.5 mm, about 14.0 mm, about 14.5 mm, or about 15 mm. The width of the distal anchor may be about 0.5 mm, about 1.0 mm, about 1.5 mm, about 2.0 mm, about 2.5 mm, about 3.0 mm, about 3.5 mm, about 4.0 mm, about 4.5 mm, or about 5.0 mm.

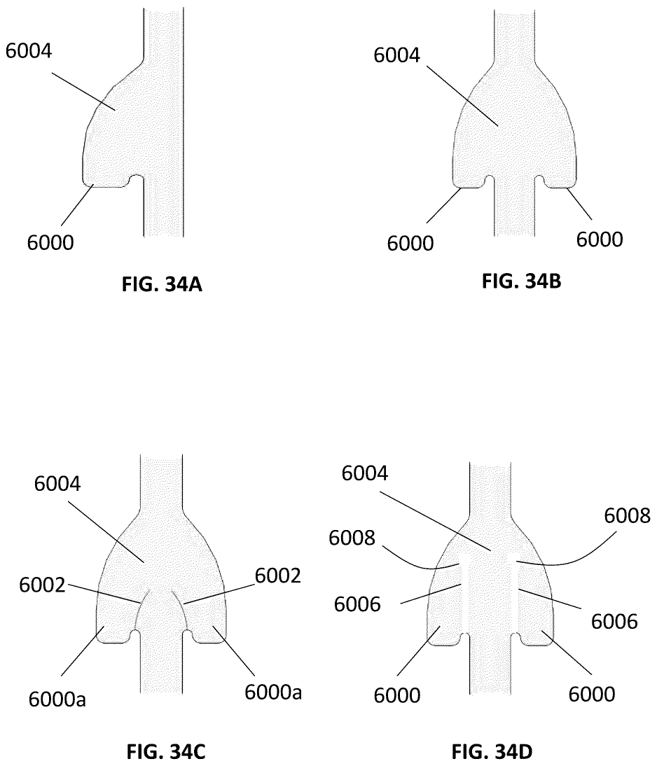

In other variations, the anchor body may include a single arm or a plurality of arms. The arm(s) may have varying shapes and geometries. In some variations, the single arm or the plurality of arms may be configured to swivel (e.g., tilt, turn, flip, rotate, pivot) about a pivot point upon the application of force to the elongate body, as described in co-owned U.S. Pat. No. 12,083,034, which is incorporated herein by reference in its entirety. Any suitable number of arms may be employed. For example, two, three, or four arms may be included. For example, referring to FIGS. 34A to 34D, the anchor body (6004) may include one arm (6000) (FIG. 34A) or two arms (6000) (FIG. 34B). When the anchor body (6004) is configured with longer arms, curved cuts (6002) may be made within the body (6004) to form curved arms (6000a) (FIG. 34C), or the arms (6000) may be formed by linear cuts (6006) terminating in a distal hole (6008) (34D). An end of one or more of the arms may have a shape configured to initiate pivoting of the distal anchor upon the application of force to the elongate body and/or facilitate its engagement against tissue. For example, the shape may include one or more of a bevel, taper, and chamfer.

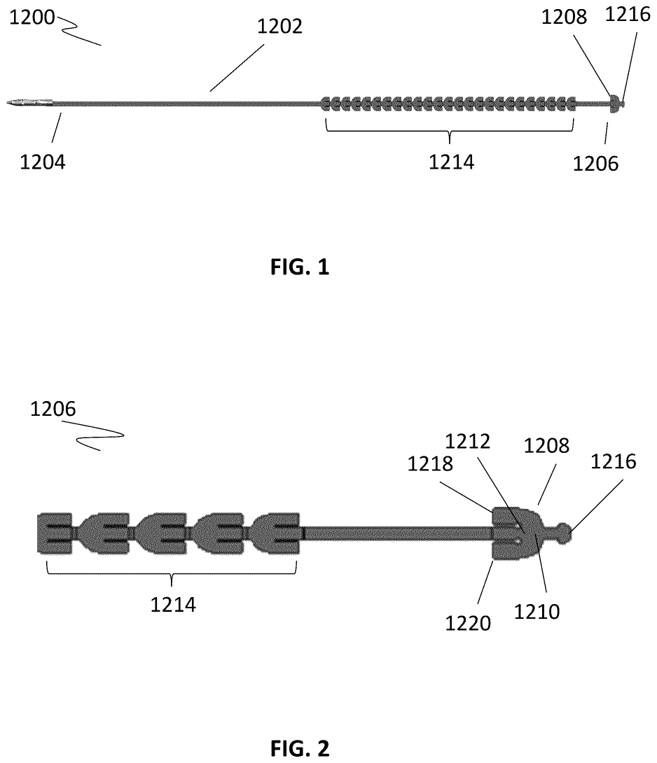

When the anchor body includes two arms and is configured to swivel (e.g., tilt, turn, flip, rotate, pivot) about a pivot point, the distal anchor (e.g., anchor 1208 in FIGS. 1 and 2) may be referred to as a “Z-Flex anchor”. The body of the Z-Flex anchor may have a width ranging from about 0.5 mm to about 5.0 mm, including all values and sub-ranges therein. For example, the Z-Flex anchor body may have a width of about 0.5 mm, about 1.0 mm, about 1.5 mm, about 2.0 mm, about 2.5 mm, about 3.0 mm, about 3.5 mm, about 4.0 mm, about 4.5 mm, or about 5.0 mm. In one variation, the Z-Flex anchor body has a width of about 2.5 mm. Furthermore, the Z-Flex anchor body may have a length ranging from about 0.5 mm to about 15 mm, including all values and sub-ranges therein. For example, the length of the Z-Flex anchor body may be about 0.5 mm, about 1.0 mm, about 1.5 mm, about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, about 8.0 mm, about 8.0 mm, about 8.5 mm, about 9.0 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, or about 15 mm. In one variation, the Z-Flex anchor body has a length of about 2.75 mm. The arms of the Z-Flex anchor body may also have a length and width. In one variation, the arm length may be about 1.5 mm and the width about 0.5 mm. The arm length may range from about 0.5 mm to about 3.0 mm, including all values and sub-ranges therein. For example, the arm length may be about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, about 2.0 mm, about 2.1 mm, ab out 2.2 mm, about 2.3 mm, about 2.4 mm, about 2.5 mm, about 2.6 mm, about 2.7 mm, about 2.8 mm, about 2.9 mm, or about 3.0 mm. The arm width may range from about 0.5 mm to about 2.0 mm, including all values and sub-ranges therein. For example, the arm width may be about 0.5 mm, about 0.6 mm, about 0.7 mm, about 0.8 mm, about 0.9 mm, about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, or about 2.0 mm. In some variations, it may be useful for the arm width to be about one third of the width of the Z-Flex anchor body. In other variations, it may be useful for the arm width to be about 70% to about 80% of the width of the tension element, including all values and sub-ranges therein, so that the distal anchor may have a holding force of greater than about 1 Newton (N). For example, it may be useful for the arm width to be about 70%, about 71%, about 72%, about 73%, about 74% about 75%, about 76%, about 77%, about 78%, about 79%, or about 80% of the width of the tension element. In one variation, the arm width may be about 0.5 mm when the tension element has a width of about 0.65 mm. In some variations, the distal anchor may provide a holding force of at least about 13 N to provide an overall tension element holding force ranging between about 5 N to about 30 N, including all values and sub-ranges therein. For example, the holding force of the distal anchor may be such that the overall force (holding force of the distal anchor and proximal anchors) of the tension element is about 5 N, about 6 N, about 7 N, about 8 N, about 9 N, about 10 N, about 11 N, about 12 N, about 13 N, about 14 N, about 15 N, about 16 N, about 17 N, about 18 N, about 19 N, about 20 N, about 21 N, about 22 N, about 23 N, about 24 N, about 25 N, about 26 N, about 27 N, about 28 N, about 29 N, or about 30 N.

Alternatively, the anchor body may be rectangular, square, triangular, circular, or ovular in shape, or in some variations, T-shaped or X-shaped. In other variations, the anchor body may be expandable from a collapsed configuration to an expanded configuration. Here the collapsed configuration may allow insertion of the distal anchor through tissue in a first direction, and the expanded configuration prevent passage of the distal anchor back through the tissue in a second direction, e.g., in a direction opposite to the first direction. In further variations, the device may include a tension element comprising an elongate body having a proximal end and a distal end, a pivot point, and a distal anchor at the distal end of the elongate body. In these variations, the distal anchor may include a first arm and a second arm having a length, where the length of the first arm from the pivot point may be different from the length of the second arm from the pivot point. For example, the first arm length may be shorter than the second arm length. The first, shorter arm length may range from about 1.0 mm to about 4.0 mm, including all values and sub-ranges therein, and the second, longer arm length may range from about 2.0 mm to about 8.0 mm, including all values and sub-ranges therein. For example, the shorter arm length may be about 1.0 mm, about 1.5 mm, about 2.0 mm, about 2.5 mm, about 3.0 mm, about 3.5 mm, or about 4.0 mm, and the longer arm length may be about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, or about 8.0 mm. The width of the distal anchor may range from about 0.5 mm to about 3.0 mm, including all values and sub-ranges therein. For example, the distal anchor width may be about 0.5 mm, about 0.75 mm, about 1.0 mm, about 1.25 mm, about 1.5 mm, about 1.75 mm, about 2.0 mm, about 2.25 mm, about 2.5 mm, about 2.75 mm, or about 3.0 mm. In some instances, it may be useful for the distal anchor to have a width of about 1.25 mm. The thickness of the distal anchor may be the same as the device thickness and range from about 0.25 mm to about 1.5 mm, including all values and sub-ranges therein. For example, the distal anchor thickness may be about 0.25 mm, about 0.5 mm, about 0.75 mm, about 1.0 mm, about 1.25 mm, or about 1.5 mm. In one variation, it may be useful for the distal anchor thickness to be about 0.65 mm. Additionally, an end of one or more of the first and second arms may have a shape configured to initiate pivoting of the distal anchor upon the application of force to the elongate body and/or facilitate its engagement against tissue. For example, the shape may include one or more of a bevel, taper, and chamfer.

A plurality of proximal anchors (e.g., migration prevention elements) may further be disposed between the distal anchor and the proximal end of the tension element. The distal anchor and the plurality of proximal anchors may be the same type of anchor or different types of anchors. In general, the proximal anchors are sized to be smaller than the distal anchor, but may be the same size if desired. Any suitable number of proximal anchors may be employed. The number of proximal anchors provided between the distal anchor and the proximal end of the elongate body may range from 2 to 40. For example, the plurality of proximal anchors may include 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 anchors. The length of the tension element including the proximal anchors may be about 75 mm.

Spacing between the plurality of proximal anchors may be the same or different. When the spacing is uniform between the plurality of proximal anchors, the length of the space may be between about 0.5 mm to about 5.0 mm, including all values and sub-ranges therein. For example, the length of the space may be about 0.5 mm, about 1.0 mm, about 1.5 mm, about 2.0 mm, about 2.5 mm, about 3.0 mm, about 3.5 mm, about 4.0 mm, about 4.5 mm, or about 5.0 mm. In one variation, the length of space between the plurality of proximal anchors may be about 1.5 mm. In some variations, the length of the space between the plurality of proximal anchors may be about 2.0 mm. In some variations, the length of the space between the plurality of proximal anchors may be about 3.0 mm. Spacing between the distal anchor and most distal proximal anchor may be between about 2.0 mm to about 10 mm, including all values and sub-ranges therein. For example, the spacing may be about 2.0 mm, about 3.0 mm, about 4.0 mm, about 5.0 mm, about 6.0 mm, about 7.0 mm, about 8.0 mm, about 9.0 mm, or about 10 mm.

In one variation, the plurality of proximal anchors may include Z-Flex anchors. Here the arms of the Z-Flex anchor may have a length ranging from about 0.25 mm to about 1.25 mm. For example, the arms may have a length of about 0.25 mm, about 0.50 mm, about 0.75 mm, about 1.0 mm, or about 1.25 mm. The arms of the Z-Flex anchor may have a width of about 0.6 mm.

In some variations, the plurality of proximal anchors may provide a holding force of at least about 6 N to provide an overall tension element holding force ranging between about 5 N to about 30 N, including all values and sub-ranges therein. For example, the holding force of the proximal anchors may be such that the overall force (holding force of distal anchor and proximal anchors) of the tension element is about 5 N, about 6 N, about 7 N, about 8 N, about 9 N, about 10 N, about 11 N, about 12 N, about 13 N, about 14 N, about 15 N, about 16 N, about 17 N, about 18 N, about 19 N, about 20 N, about 21 N, about 22 N, about 23 N, about 24 N, about 25 N, about 26 N, about 27 N, about 28 N, about 29 N, or about 30 N.

An enlarged tip (toe) may also be provided at the distal end of the tension element distal to the distal anchor to further facilitate anchoring of the tension element to tissue and/or coupling to an anchor delivery element, as further described below. The distal anchor, enlarged tip, and plurality of proximal anchors may be made from the same materials as the tension element, or from different materials. In some variations, the distal anchor may be made from a non-biodegradable material, and the plurality of proximal anchors made from a biodegradable material, for example, a biodegradable polymer.

The tension element, distal anchor, enlarged tip, and plurality of proximal anchors may be provided with a coating. In some variations, the coating may include an antibacterial agent. Exemplary antibacterial agents include without limitation, aminoglycosides, amphenicols, ansamycins, betalactams, β-lactams such as penicillins, lincosamides, macrolides, nitrofurans, quinolones, sulfonamides, sulfones, tetracyclines, vancomycin, and any of their derivatives, or combinations thereof. Examples of penicillins that can be suitable for use with the described methods and devices include, but are not limited to, amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, 28longate28in, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, and ticarcillin.

In other variations, the coating may include growth factors that promote cartilage remodeling. Exemplary growth factors include without limitation, TGF-β1 (transforming growth factor-β), BMP-2 (bone morphogenetic protein-2), BMP-7 (bone morphogenetic protein-7), IGF-I (insulin growth factor-I), FGF-2 (fibroblast growth factor-2), FGF-18 (fibroblast growth factor-18), and PDGF (platelet-derived growth factor).

In further variations, the coating may include a hydrophobic polymer to slow the degradation of the tension element. Examples of hydrophobic polymers that may be used to form the coating include, but are not limited to, fluoropolymers such as polytetrafluoroethylene (PTFE) and expanded polytetrafluoroethylene (ePTFE), polyvinyl chloride (PVC), polyvinylacetate, poly(ethylene terephthalate), silicone, polyesters, polyamides, polyureas, styrene-block copolymers, polymethyl methacrylate, acrylic-butadiene-styrene copolymers, polyethylene, polystyrene, polypropylene, natural and synthetic rubbers, acrylonitrile rubber, and mixtures and copolymers of any of the foregoing.

In yet further variations, the coating may include a vasoconstrictive agent. Examples of vasoconstrictive agents include without limitation, epinephrine, levonordefrin, and adrenaline. In some variations, the coating may include a decongestant. Exemplary decongestants include, but are not limited to, epinephrine, pseudoephedrine, oxymetazoline, phenylephrine, tetrahydrozolidine, and xylometazoline. The coating may also include an anti-inflammatory agent. Exemplary anti-inflammatory agents include, but are not limited to, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, any of their derivatives, and combinations thereof.

### Needles and Anchor Delivery Elements

The devices described herein may further include a proximal needle removably attached to the proximal end of the elongate body of the tension element. The proximal needle may be a cutting needle having a length ranging from about 5.0 mm to about 25 mm, including all values and sub-ranges therein. For example, the proximal needle may have a length of about 5.0 mm, about 10 mm, about 15 mm, about 20 mm, or about 25 mm. In one variation, the proximal needle has a length of about 13 mm. Proximal needle diameters may range from about 0.4 mm to about 2.0 mm, including all values and sub-ranges therein. For example, the proximal needle diameter may be about 0.4 mm, about 0.5 mm, about 1.0 mm, about 1.5 mm, or about 2.0 mm. In one variation, the proximal needle diameter is about 1.0 mm. In another variation, the proximal needle diameter is about 1.5 mm.

The proximal needle may be used to place or manipulate the proximal end of the elongate body through or around tissue, and may be removably attached in various ways to the tension element. For example, the proximal needle may be removable attached to the tension element by swaging or crimping, or by threading the tension element through a portion of the proximal needle. In some variations, the proximal needle is a quick-thread needle. In other variations, the proximal needle may be swaged to a loop of material, e.g., PDO (Poly(dioxanone)), which may then be coupled to the tension element.

At the distal end of the tension element, an anchor delivery element may be coupled to the distal anchor. The anchor delivery element may include a cutting tip configured to pass the distal anchor through the tissue in its insertion configuration. The anchor delivery element may be made from various metals, including but not limited to, stainless steel, spring steel, and nitinol.

In some variations, the anchor delivery element may include a tip component configured to cut and/or pierce tissue and an anchor support. The tip component may include a cutting tip, and one or more of a cockpit or pocket shaped to removably secure the enlarged distal end of the tension element (toe), a clip region that removably secures the region of the tension element between the toe and distal anchor to the anchor delivery element, and a seating region upon which the distal anchor may be positioned prior to deployment. In one variation, the tip component and the anchor support may comprise different components that are joined to form the anchor delivery element. In other variations, the tip component and anchor support may be integrally formed as a single piece. Materials that may be used to make the tip component and the anchor support include without limitation, stainless steel, spring steel, and nitinol. The tip component and anchor support may comprise the same metal material or different metal materials. For example, in some variations, the tip component may be made from stainless steel, and the anchor support made from nitinol.

The cutting tip of the anchor support may have varying amounts of curvature that may help a user more easily track the cutting tip against tissue and initiate a puncture within the tissue (providing a tactile advantage to the user), as previously described. In these variations, the vertex of the angle of curvature may be located at the distal end of the distal tip or at the proximal end of the distal tip. When the vertex is located at the distal end of the distal tip, the angle of curvature may range from about 15 degrees to about 45 degrees, including all values and sub-ranges therein. For example, the angle of curvature may be about 15 degrees, about 20 degrees, about 25 degrees, about 30 degrees, about 35 degrees, about 40 degrees, or about 45 degrees. In some instances, it may be useful for the angle of curvature to be about 20 degrees. In other instances, it may be useful for the angle of curvature to range from about 10 degrees to about 30 degrees. When the vertex is located at the proximal end of the distal tip, the angle of curvature may range from about 10 degrees to about 45 degrees, including all values and sub-ranges therein. For example, the angle of curvature may be about 10 degrees, about 15 degrees, about 20 degrees, about 25 degrees, about 30 degrees, about 35 degrees, about 40 degrees, or about 45 degrees. The distal cutting tip may also have a sharpness and/or shape that allows the user to more easily track the distal tip against tissue and initiate a puncture within the tissue (providing a tactile advantage to the user).

When the tip component and anchor support are separate components, they may be joined to form the anchor delivery element via one or more rivets. Alternatively, the tip component and anchor support may be joined by crimping, welding, or riveting. The tip component may be made by processes such as laser sintering, injection molding, or machining. Furthermore, the tip component may be formed such that it or the distal anchor does not have any leading edges that may catch on tissue during delivery to a target tissue. For example, the anchor delivery element and distal anchor of the tension element may form a level surface that may prevent the distal anchor from catching on tissue during insertion. In some variations, the seating region may include an area of decreased thickness in order to level the surface of the distal anchor with the anchor support when the distal anchor is seated on the anchor delivery element. In other variations, the anchor support may not include an area of decreased thickness.

The tension elements described herein may have various configurations. Referring to FIG. 1, an exemplary tension element is shown. Tension element (1200) may include an elongate body (1202) having a proximal end (1204) and a distal end (1206). A distal anchor, such as Z-Flex anchor (1208), may be disposed at the distal end (1206) of the elongate member (1202). As shown in the enlarged view of the distal end (1206) in FIG. 2, the tension element may include a Z-Flex anchor (1208) having an anchor body (1210), a pivot point (1212), and a first arm (1218) and a second arm (1220). A plurality of proximal anchors (1214) may also be disposed between the distal Z-Flex anchor (1208) and the proximal end (1204) of the tension element (1202) to help prevent migration of the tension element (1200) once deployed in a tissue. The plurality of proximal anchors (1214) may also be Z-Flex anchors that are facing in a direction opposite to that of the distal Z-Flex anchor (1208). An enlarged distal end (toe) (1216) may also be provided distal to the Z-Flex anchor (1208) to couple the Z-Flex anchor to an anchor delivery element (not shown) for deployment into tissue. At the proximal end (1204), a needle (1201) may be removably attached to the elongate body (1202). After deployment of the Z-Flex anchor into tissue, the needle (1201) may be used to place or manipulate the proximal end (1204) of the elongate body (1202) through or around tissue.

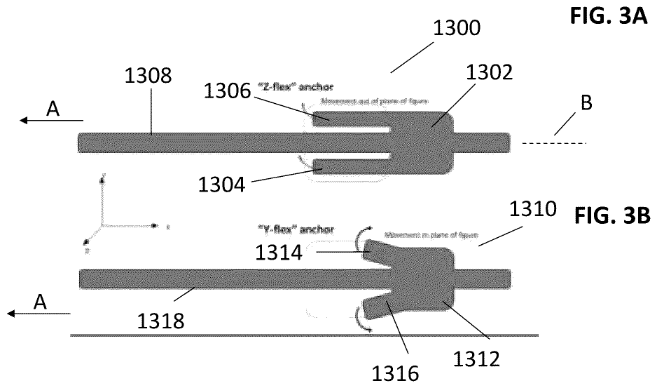

Once inserted into tissue, the application of a force to the elongate body of the tension element may swivel (e.g., tilt, turn, flip, rotate, or pivot) the arms of the Z-Flex anchor to transform the anchor from the insertion configuration to the deployed configuration. As shown in FIG. 3A, Z-Flex anchor (1300) includes an anchor body (1302), a first arm (1304), and a second arm (1306). Upon the application of force to the tension element (1308) in the direction of arrow A, which is in a direction opposite to the direction of device insertion, the first and second arms (1304, 1306) swivel (e.g., tilt, turn, flip, rotate, or pivot) out of plane along the z-axis and orthogonal to the axis (B) of the tension element. In the deployed configuration, passage of the Z-Flex anchor (1300) back through tissue is prevented. In some variations, each arm of the plurality of arms of the anchor body may include a distal end that is bevel cut to form a slope that may help swivel the distal anchor at the pivot point and facilitate its engagement against tissue. In other variations, one arm of the plurality of arms may be bevel cut.

In another variation, as shown in FIG. 3B, the distal anchor, Y-Flex anchor (1310), includes an anchor body (1312) having a first arm (1314) and a second arm (1316). However, instead of swiveling out of plane upon the application of force to the tension element (1318) in the direction of arrow (A), the first and second arms (1314, 1316) flex in plane to prevent passage of the Y-Flex anchor (1310) back through tissue.

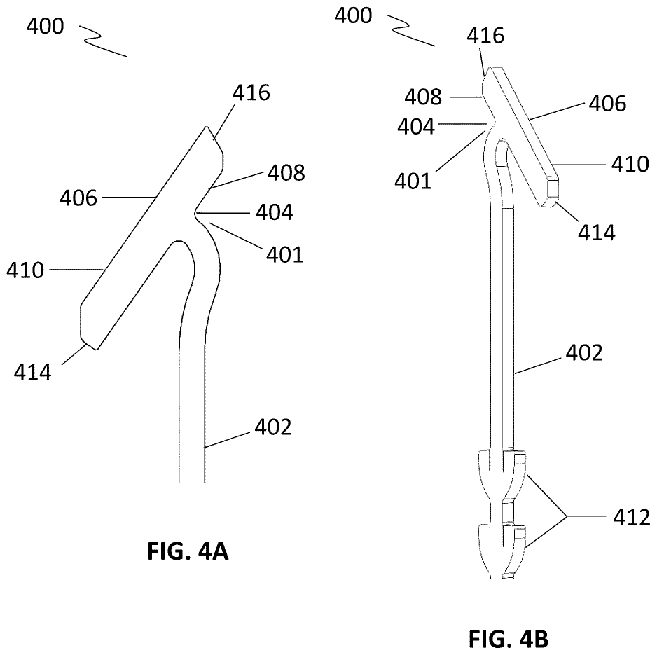

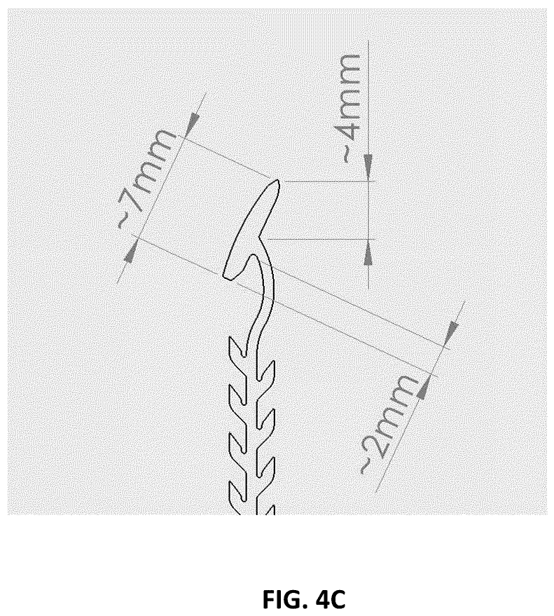

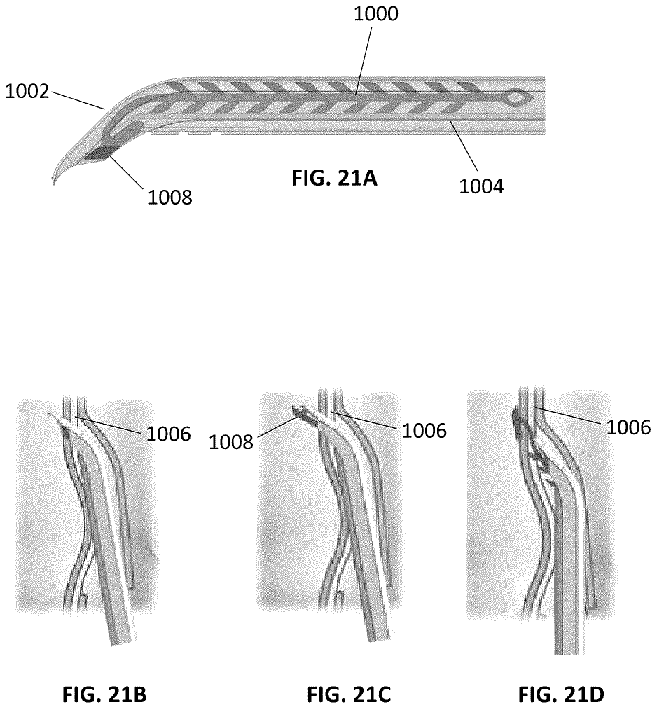

In further variations, the device may include a tension element comprising an elongate body having a proximal end and a distal end, a pivot point, and a distal anchor at the distal end of the elongate body. In these variations, the distal anchor may include a first arm and a second arm having a length, where the length of the first arm from the pivot point may be different from the length of the second arm from the pivot point. During a minimally invasive procedure, the distal anchor may be advanced through tissue and a force applied to the proximal end of the elongate body such that the distal anchor transitions from an insertion configuration to a deployed configuration by swiveling (e.g., tilting, turning, flipping, rotating, or pivoting) about the pivot point. For example, referring to FIGS. 4A and 4B, a tension element may include an elongate body (402), and a pivot point (404) and a distal anchor (406) at a distal end (401) of the elongate body (402). The distal anchor (406) may include a first arm (408) and a second arm (410) configured such that the first arm (408) has a length from the pivot point (404) that is shorter than a length of the second arm (410) from the pivot point (404). Additionally, ends (414, 416) may be chamfered to help transition the distal anchor between the insertion configuration and the deployed configuration by swiveling (e.g., tilting, turning, flipping, rotating, or pivoting) about the pivot point (404). In the deployed configuration (see, e.g., FIG. 21D), the first and second arms (408, 410) of the distal anchor (406) may lie flat against the tissue. When the first and second arms have different lengths, they may be configured as shown in FIG. 4C. Referring to FIG. 4C, the distal anchor body may have a length of about 7.0 mm; the first, shorter arm length may be about 2.0 mm; and the second, longer arm length may be about 4.0 mm. The arms may have other shorter and longer lengths, as previously described herein.

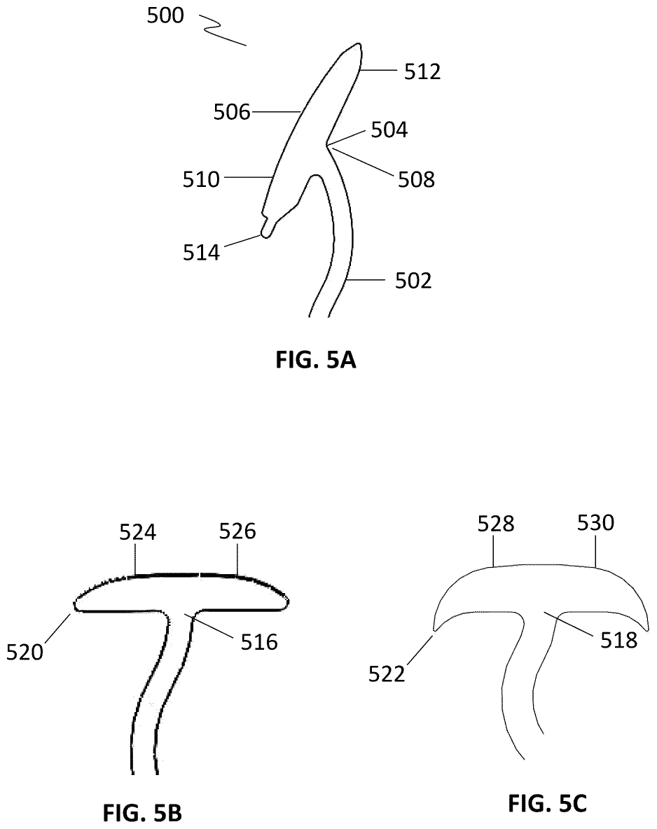

In another variation, as shown in FIG. 5A, the distal anchor (500) may include an elongate body (502), a pivot point (502), and a distal anchor (506) at a distal end (508) of the elongate body (502). The distal anchor (506) may include a first arm (510) and a second arm (512) configured such that the first arm (510) has a length from the pivot point (504) that is shorter than a length of the second arm (512) from the pivot point (504). Additionally, an end of the first arm (510) may include a tab or protrusion (514) that may help swivel (e.g., tilt, turn, flip, rotate, or pivot) the distal anchor (506) about the pivot point (504) to facilitate apposition and flattening of both the first and second arms (510, 512) against tissue. In further variations, as shown in FIGS. 5B and 5C, the first arms (524, 528) and second arms (526, 530) may have the same length from the pivot point (516, 518). One or more ends of the arms may be blunt, rounded, or curved (520), or sharp, pointed, or tapered (522).

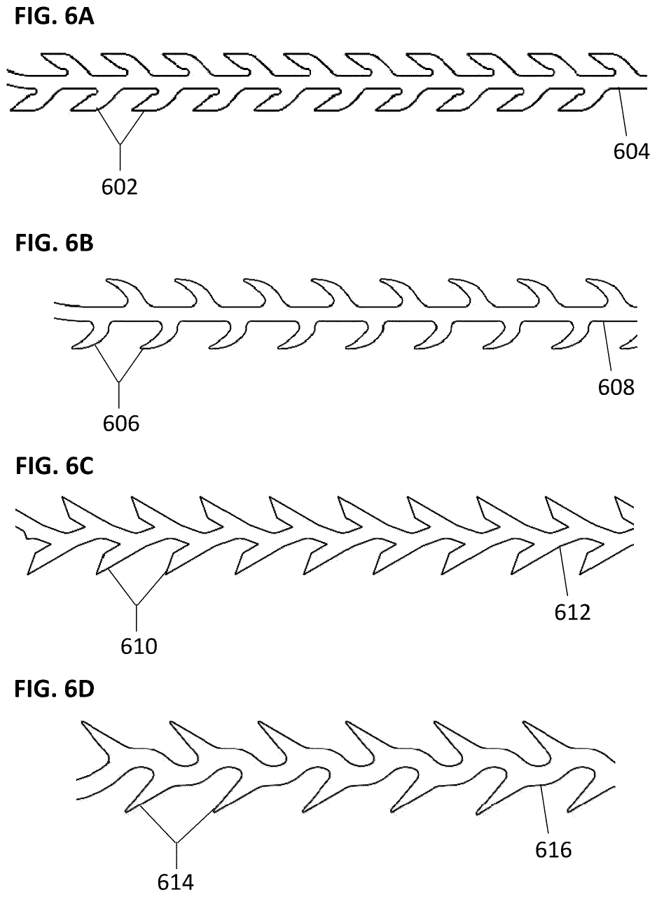

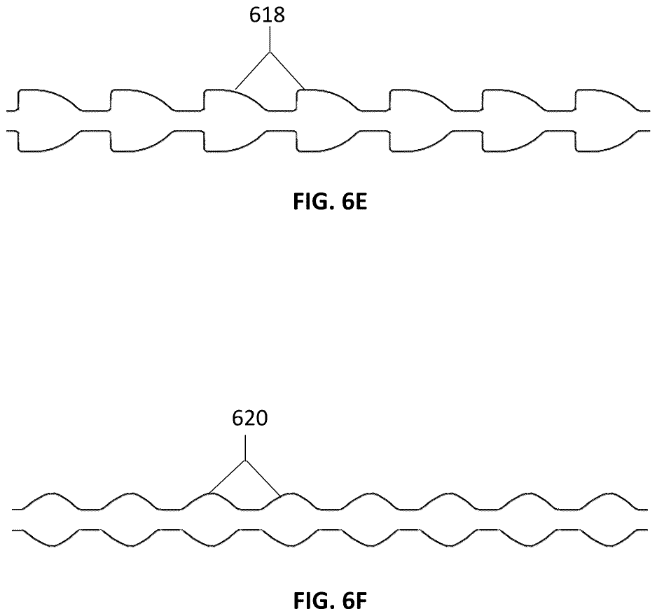

The tension elements described herein may also include one or more proximal anchors spaced along a portion of the length of the elongate body. When a plurality of proximal anchors is employed, the tension elements may be configured as shown in FIGS. 6A-6F. For example, the plurality of proximal anchors may be staggered along a length of the elongate body of the tension element. An end (e.g., the free end) of each of the plurality of proximal anchors may be beveled (602, 610 in FIGS. 6A and 6C), hooked (606 in FIG. 6B), or pointed (614 in FIG. 6D). The elongate body may also be configured to be straight (604, 608 in FIGS. 6A and 6B) or undulating (612, 616 in FIGS. 6C and 6D). In some variations, the proximal anchors may be shaped like a shield (618 in FIG. 6E) or have a bulbous shape (620 in FIG. 6F). Although the proximal anchors are shown as symmetrically spaced apart in FIGS. 6A-6F, they may be spaced differently from each other along the length of the elongate body (e.g., asymmetrically spaced apart). In further variations, the proximal anchors may have a reduced cross-section at its root to allow them to flex inwards while traversing cartilage, minimizing tissue trauma caused by the passing anchors.

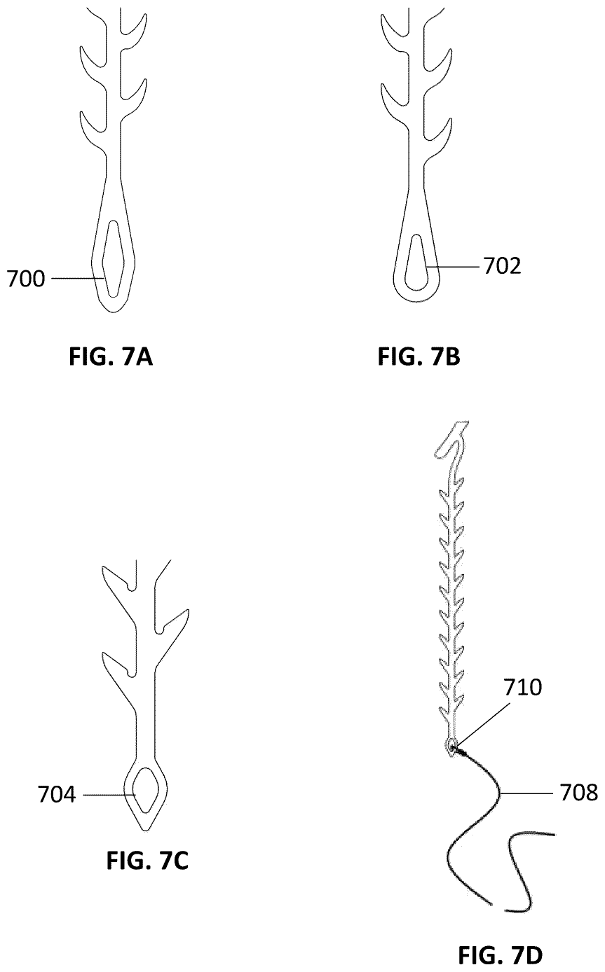

Some variations of the tension element, as shown in FIGS. 7A-7C, may include an eyelet at the proximal end of the elongate body. The eyelet may be variously shaped. For example, the eyelet may be diamond shaped (700 in FIG. 7A), teardrop shaped (702 in FIG. 7B), ovular in shape (704 in FIG. 7C), or other shapes such as circular, triangular, rectangular, or square. A suture (708) may be coupled to an eyelet (710) as shown in FIG. 7D to provide increased length at the proximal end of the tension element.

A proximal needle may be used to place or manipulate the proximal end of the elongate body through or around tissue, and may be removably attached in various ways to the tension element. For example, the proximal needle may be a quick-thread needle designed to allow an operator to manually connect the proximal end just prior to insertion into the patient.

At the distal end of the tension element, an anchor delivery element may be coupled to the distal anchor. The anchor delivery elements may have various configurations, and may be reversibly secured to the tension element in various ways. In general, the anchor delivery element may include a cutting tip configured to pass the distal anchor through the tissue in its insertion configuration.

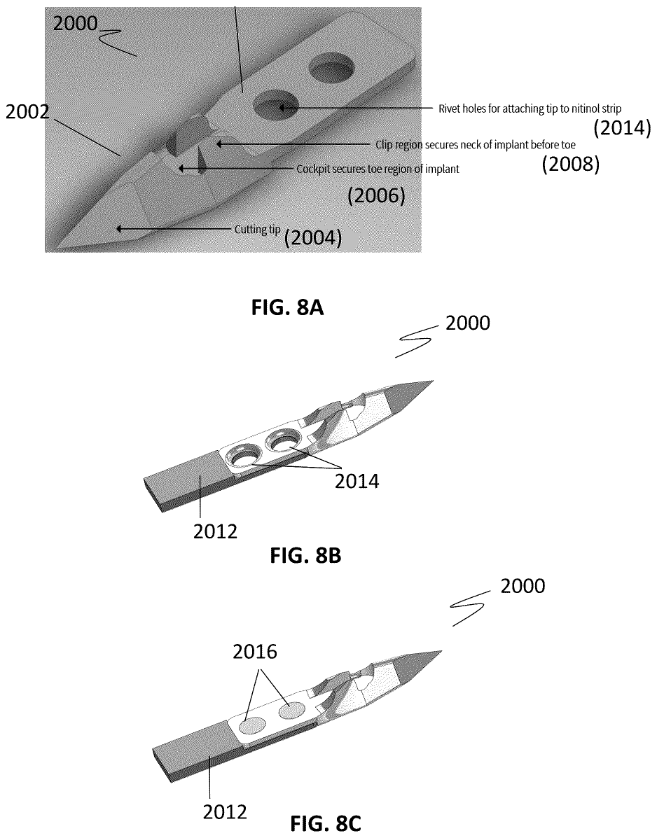

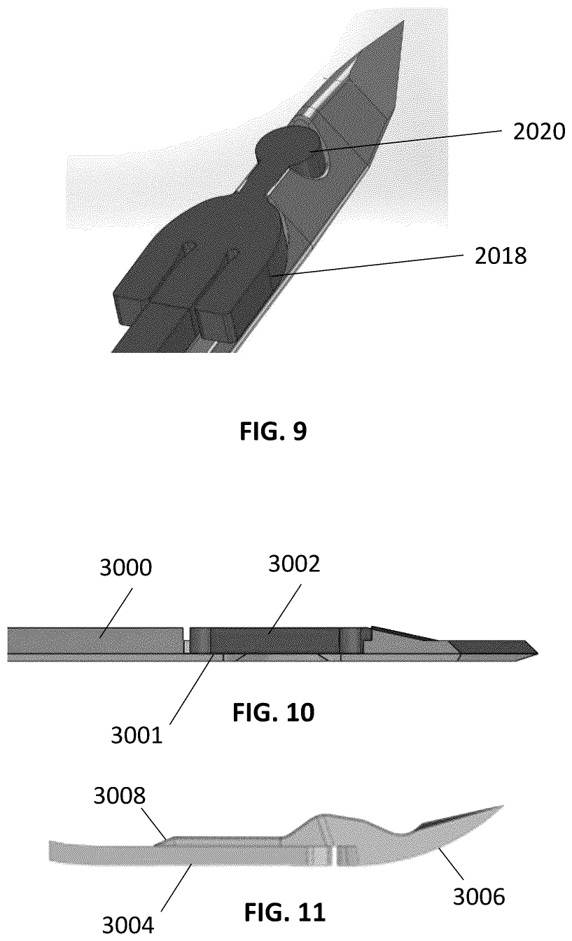

In other variations, the anchor delivery element may include a tip component and an anchor support. Referring to FIGS. 8A-8C, the tip component (2002) of an anchor delivery element (2000) may include a cutting tip (2004), a cockpit or pocket (2006) shaped to removably secure the enlarged distal end of the tension element (toe) (e.g., see element 2020 in FIG. 9), a clip region (2008) that removably secures the region of the tension element between the toe and distal anchor to the anchor delivery element, and a seating region (2010) on the anchor support upon which the distal anchor may be positioned prior to deployment. FIG. 9 shows an exemplary Z-Flex anchor (2018) seated within the anchor delivery element described in FIGS. 8A-8C.

In one variation, the tip component and the anchor support may be integrally formed as a single piece. In another variation, the tip component (2000) and the anchor support (2012) may be separate components that are joined together, as shown in FIGS. 8A and 8B. In this variation, the tip component (2000) includes rivet holes (2014) that may align with corresponding rivet holes in the anchor support (2012). Rivets (2016) may be placed in the rivet holes to secure the tip component (2000) and anchor support (2012) together to form the anchor delivery element. The tip component and anchor support may also be joined by crimping or welding.

The tip component may be configured in various ways. In one variation, the tip component may be formed such that it or the distal anchor does not have any leading edges that may catch on tissue during delivery to a target tissue. For example, as shown in FIG. 10, the anchor delivery element (3000) and distal anchor (3002) of the tension element (not shown) may form a level surface that may prevent the distal anchor from catching on tissue during insertion. The level surface may be provided by a portion of the anchor support (e.g., the seating region 3001) of the anchor delivery element (3000) having a decreased thickness. However, in other variations, as shown in FIG. 11, the anchor support (3004) may be full thickness along its length and not include an area of decreased thickness. In these variations, the cutting tip (3006) may be attached (e.g., using an adhesive, welding, other types of bonding) on the full thickness anchor support (3004), and a bevel (3008) on the proximal end of the cutting tip (3006) used to help prevent snagging on tissue. The use of a full thickness anchor support may help to improve pushability of the anchor delivery element through tissue.

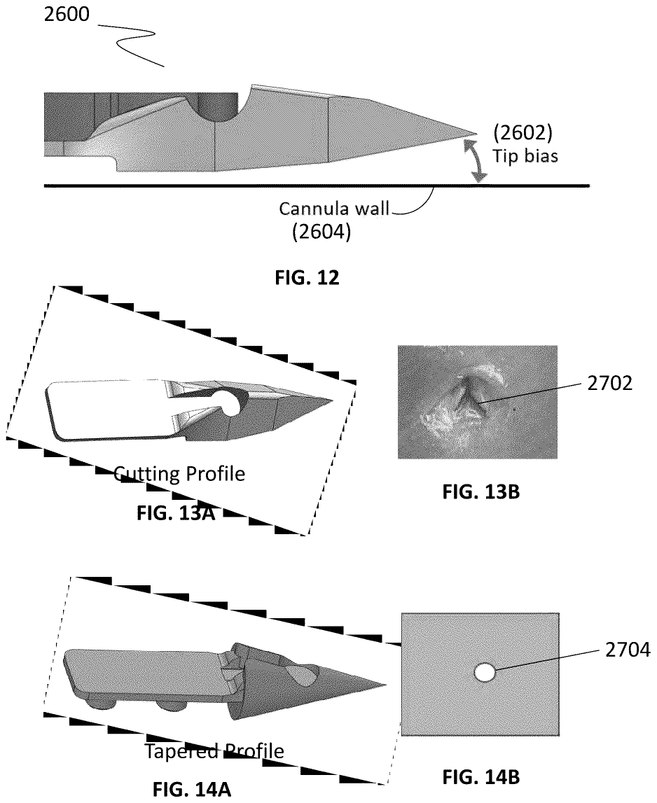

Referring to FIG. 12, the cutting tip component (2600) may also be configured to include a tip bias (2602) from a cannula wall (2604) to help facilitate travel through tissue such as cartilage.

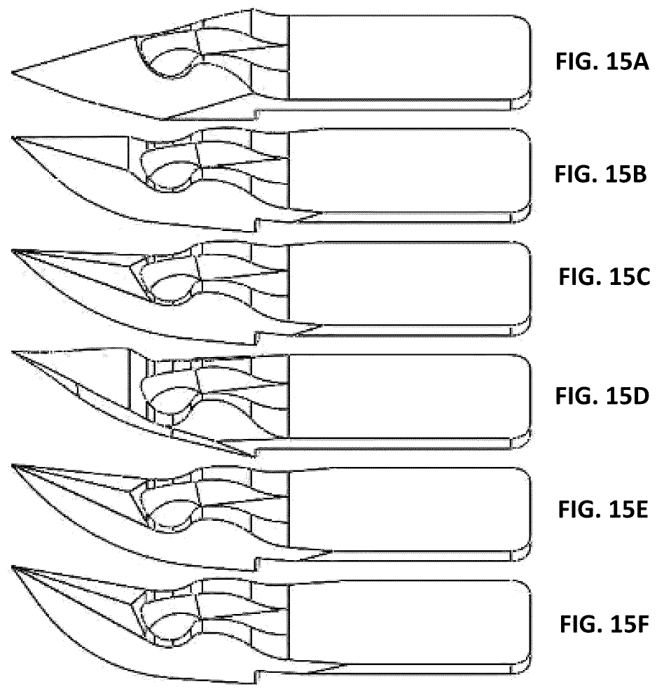

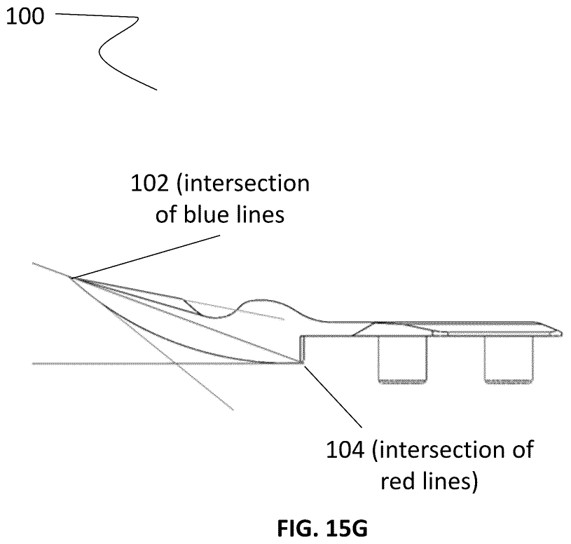

As shown in FIG. 12, the tip bias (2602) may be about 20 degrees. However, in other variations, the tip bias may range from about 5 degrees to about 35 degrees from the cannula wall (2604) when loaded within a cannula. In one variation, the tip bias may be configured to match the bend angle or the curvature of the cannula. Other tip component variations may include a cutting profile, as shown in FIG. 13A, which may result in a tri-leaflet defect (2702) in cartilage, as illustrated in FIG. 13B. A tri-leaflet defect is non-coring (e.g., does not remove a large amount of tissue), and thus may reduce the risk of septal perforation when working with nasal septal cartilage and may improve healing of the tissue. Alternatively, the tip component may have a tapered profile, as shown in FIG. 14A, which may result in a circular defect (2704) in cartilage, as depicted in FIG. 14B. The configuration of defects may vary, but in general, it may be beneficial for the width of the distal anchor to be larger than the defect width to help fix the distal anchor within the target tissue. Other curved, cutting tip component configurations, which may be useful when full thickness anchor supports are employed, are shown in FIGS. 15A-15G. Referring to FIG. 15G, the distal cutting tip (100) may have a distal end (102), a proximal end (104), and an angle of curvature measured at a first vertex (intersection of blue lines) located at the distal end (102). This angle of curvature may range from about 15 degrees to about 45 degrees, including all values and sub-ranges therein, as previously described. It may be useful for the angle of curvature at the first vertex to be about 30 degrees. Alternatively, the angle of curvature may be measured at a second vertex (intersection of red lines) located at the proximal end (104). This angle of curvature may range from about 10 degrees to about 45 degrees, including all values and sub-ranges therein, as previously described. It may be useful for the angle of curvature at the second vertex to be about 20 degrees. The tip component may be made from various materials, for example, polymers or metals. The polymer may be any biodegradable or a non-biodegradable polymer described herein. Exemplary metals include, but are not limited to, silver, platinum, stainless steel, nickel, titanium, and alloys thereof. The tip components may be made in various ways, for example, by injection molding, three-dimensional (3D) printing, or machining (e.g., electrical discharge machining (EDM) or computer numerical control (CNC) machining).

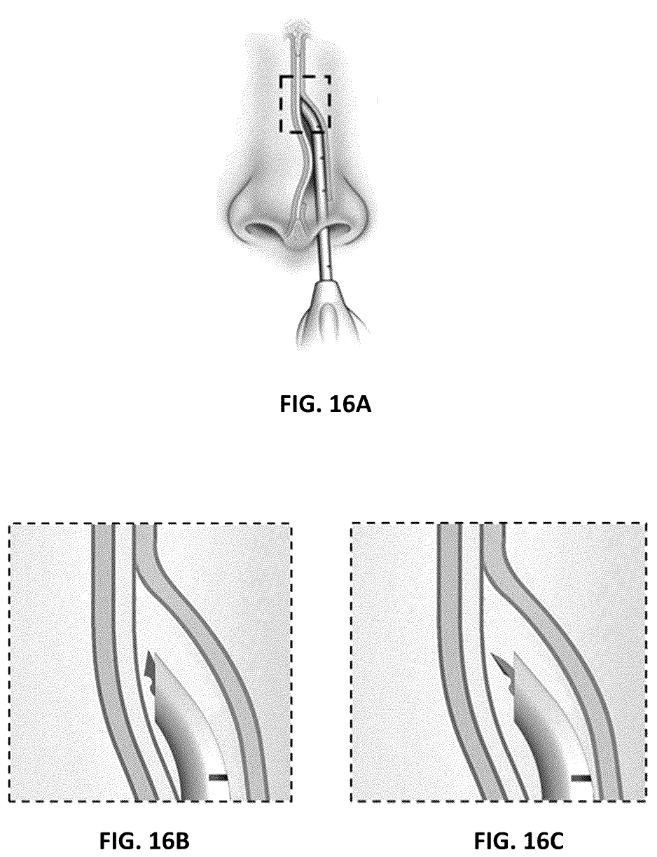

In some variations, as illustrated in FIGS. 16A to 16C, a curved cutting tip (e.g., an upwardly curved cutting tip) may help to reduce technique dependence of tension element delivery by increasing the propensity of the cutting tip to engage the tissue (e.g., cartilage) rather than skidding ipsilaterally, and increasing retention of the distal anchor within the anchor delivery device during tissue puncture (e.g., by providing better shielding with the upwards curve) without impacting release mechanism.

### Delivery Devices/Systems

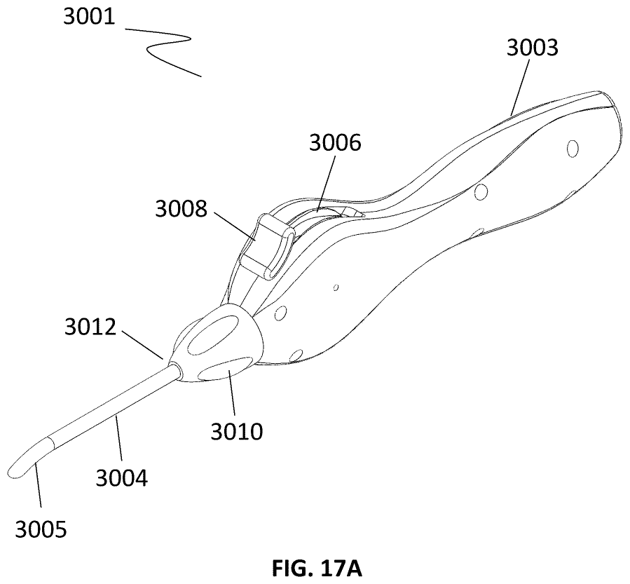

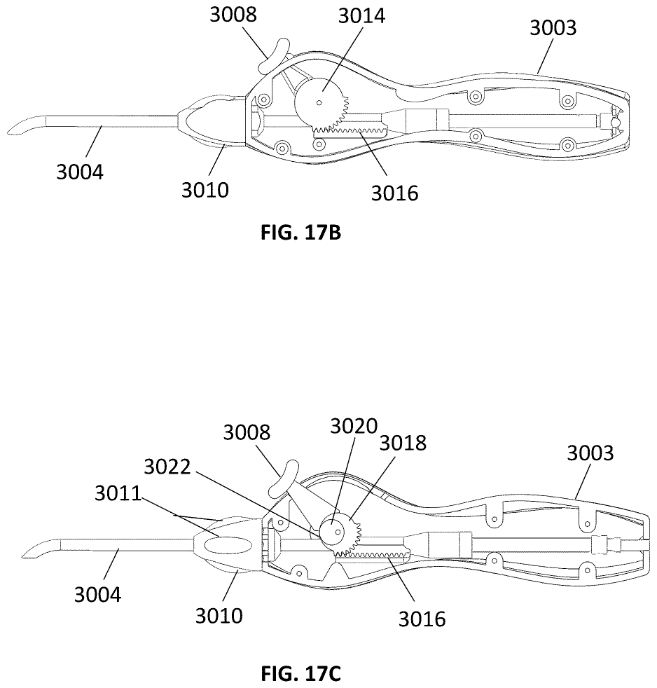

The anchor delivery elements shown in FIGS. 8A-8C and 9-11 may be advanced using the delivery devices described in co-owned U.S. Pat. No. 12,083,034, which is incorporated herein by reference in its entirety. As used herein, the terms “delivery device,” “delivery system,” and “system” are used interchangeably throughout. For example, the delivery devices may include a mechanism or actuator configured to provide mechanical advantage to the deployment of the anchor delivery element from the cannula. Delivery devices with improved mechanical advantage may improve the ability of the device to puncture bone or thick or ossified cartilage, may improve the ease of tension element deployment for the physician/surgeon, and/or may help reduce the pressure applied to brace the delivery device (and thus reduce the discomfort felt by an awake patient). The mechanical advantage may range from about 2:1 to about 4:1, including all values and sub-ranges therein. For example, the mechanical advantage may be about 2:1, about 2.5:1, about 3:1, about 3.5:1, or about 4:1. Such mechanisms may include linear gears and circular gears, e.g., rack and pinion mechanisms. The mechanisms may also include a capstan and a bowstring. An exemplary rack and pinion mechanism for the delivery device shown in FIG. 17A is illustrated in FIGS. 17B and 17C. In FIG. 17A, delivery device (3001) includes a handle (3003) and a cannula (3004). Although the cannula (3004) is shown as having a curved distal end (3005), it is understood that the distal end may also be straight. The handle (3003) may include a slot (3006) through its surface so that a finger tab (3008) may be moved back and forth to actuate a pinion, as further described below. A knob (3010) may be attached to the proximal end (3012) of the cannula (3004) and be configured to rotate the cannula when rotated. Additionally, one or more indicators may be included on the surface of the knob (3010) to help identify the orientation of the curve when the delivery device includes a curved cannula. The indicator may be a detent, recess, nub, fin, or other protuberance extending from the surface of the knob. For example, referring to FIG. 17C, the one or more indicators may be fins (3011).

In one variation, as shown in FIG. 17B, the pinion may be a fixed sector gear (3014) configured to engage a corresponding rack (3016). In use, when the finger tab (3008) is moved back or forward in the slot (3006) (see FIG. 17A), the fixed sector gear (3014) that is coupled thereto rotates, which in turn linearly moves the rack (3016) to advance or retract the anchor delivery element (not shown). In another variation, as shown in FIG. 17C, the pinion may comprise an eccentric gear (3018). The eccentric gear (3018) may include a central pin (3020) that rides within a channel (3022) of the handle (3003). When the finger tab (3008) is moved back or forward in the slot (3006) (see FIG. 17A), the eccentric gear (3014), which is coupled to the finger tab (3008), also rotates, which in turn linearly moves the rack (3016) to advance or retract the anchor delivery element (not shown). Use of the eccentric gear (3018) may help maximize initial cartilage puncture and also enhance travel of the anchor delivery element. The rack and pinion mechanisms of FIGS. 17B and 17C may provide a mechanical advantage of about 2:1 to about 4:1.

The tissues that may be manipulated with the devices describe herein include without limitation, nasal tissues, throat tissues, and ear tissues. Non-limiting examples of nasal tissue include nasal septal cartilage, lateral nasal cartilage, major alar cartilage, minor alar cartilage, alar fibrofatty tissue, nasal bone, or a nasal turbinate. Exemplary throat tissues include without limitation, oropharyngeal soft tissue, the uvula, soft palate, and tonsils. Non-limiting examples of ear tissues include cartilage of the helix, anti-helix, tragus, anti-tragus, superior crus, fossa triangularis, concha, and connective tissue of the earlobe. Systems for reshaping tissues are also described herein. The systems may generally include a pusher and a device including a tension element. The pusher may include a proximal end, a distal end, a length therebetween, and a connector at the distal end. The tension element may include an elongate body, a pivot point, and a distal anchor configured to releasably couple to the connector of the pusher. As previously described, the distal anchor may include an anchor body and first and second arms having a length. The length of the first arm from the pivot point may be different from the length of the second arm from the pivot point. For example, the first arm length may be shorter than the second arm length. The device may include a plurality of proximal anchors disposed between the distal anchor and the proximal end of the elongate body of the tension element, as also previously described.

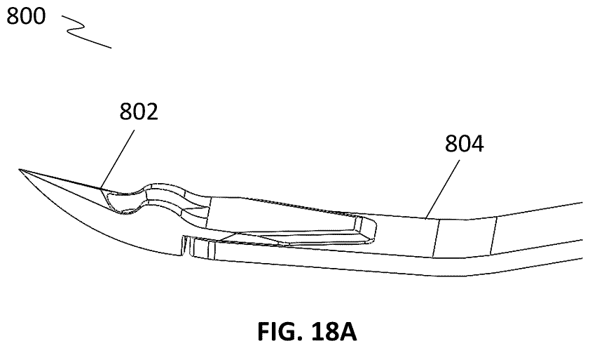

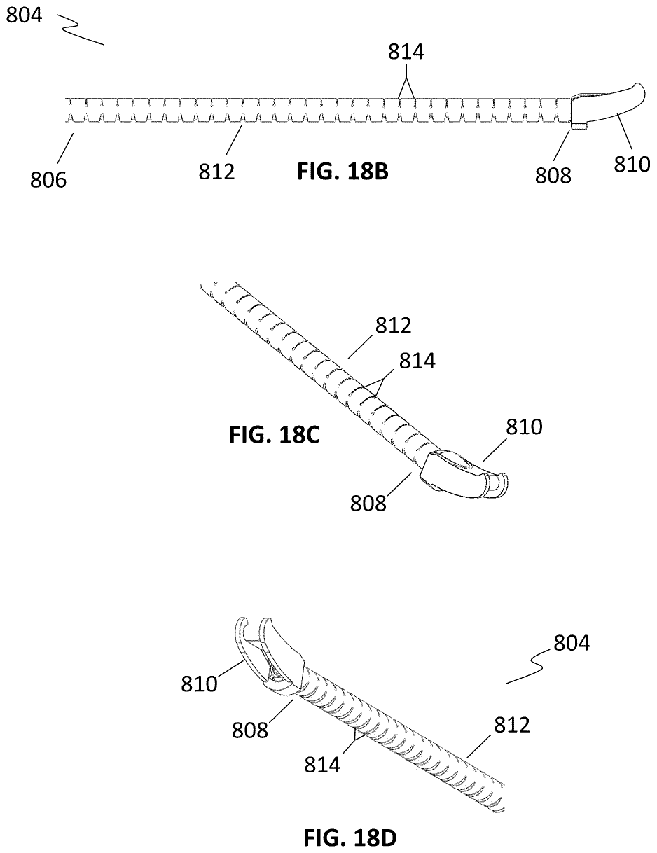

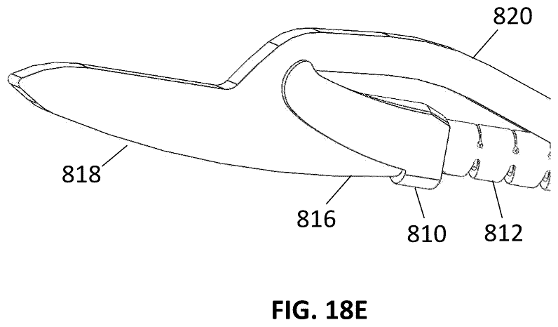

The pusher of the system may include a plurality of slits along its length. The plurality of slits may be symmetrically or asymmetrically spaced apart along the length of the pusher. For example, referring to FIG. 18A, the anchor delivery device (800) may include a cutting tip (802) and a pusher (804). Referring to FIGS. 18B to 18D, the pusher (804) may include a proximal end (806), a distal end (808), a length (body 812) therebetween, and a connector (at the distal end (808). As shown in FIG. 18E, one arm (816) of a distal anchor (818) may be releasably coupled to the connector (810) of the pusher. The pusher (804) may also include a plurality of slits (814) along its length. The plurality of slits (814) may be symmetrically or asymmetrically spaced apart along the length (812) of the pusher (804).

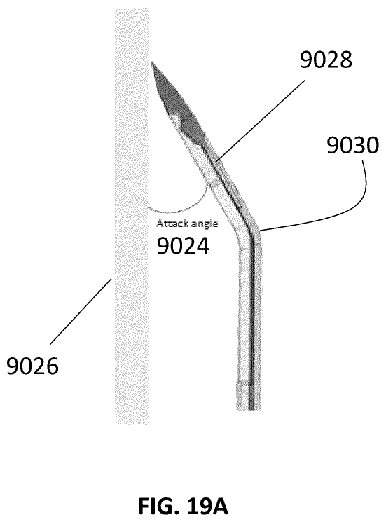

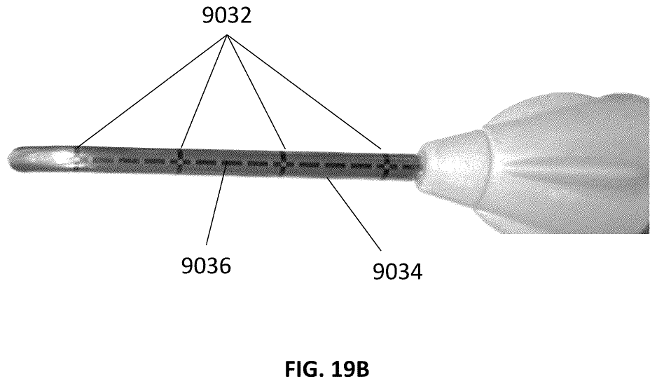

Some variations of the system may further include a cannula through which the tension element is advanced for deployment into tissue. The cannula may include a distal end having an angle of curvature configured to decrease the amount of force applied to advance a distal tip of the cannula through tissue (providing a mechanical advantage to the user). For example, as shown in FIG. 19A, the angle (9024) between tissue (e.g., nasal cartilage 9026) and the portion of the cannula (9028) distal to the curve (9030) may range from about 10 degrees to about 60 degrees, including all values and sub-ranges therein. For example, the angle (9024) may be about 10 degrees, about 15 degrees, about 20 degrees, about 25 degrees, about 30 degrees, about 35 degrees, about 40 degrees, about 45 degrees, about 50 degrees, about 55 degrees, or about 60 degrees. The angle employed may help deploy the distal anchor at the target location.

The cannula length may range from about 50 mm to about 70 mm, including all values and sub-ranges therein. For example, the length of the cannula may be about 50 mm, about 55 mm, about 60 mm, about 65 mm, or about 70 mm. In some variations, one or more portions along the cannula length may be flexible or malleable. In other variations, one or more markers may be provided along the cannula to help with visualizing the distal end of the cannula and/or determining the length of cannula inserted into the nasal cavity or tissue. In some variations, the markers may be laser markings. For example, as shown in FIG. 19B, a first set of laser markings (9032) spaced approximately 1.0 cm apart along the length of the cannula (9034) may be included to help determine the length of the cannula (9034) that has been inserted into the nasal cavity or tissue. Another centerline laser marking (9036) may be included to help with rotational orientation of the cannula (9034) prior to deployment of the tension element. A light element, imaging element, or visualization element may also be included in portion of the system to help with visualization. In some variations, the light element may be a light wire configured to slide within the cannula lumen or a second lumen concentrically disposed within the cannula lumen. Exemplary imaging and visualization elements include without limitation, a fiberoptic visualization device, CCD, CMOS or other camera.

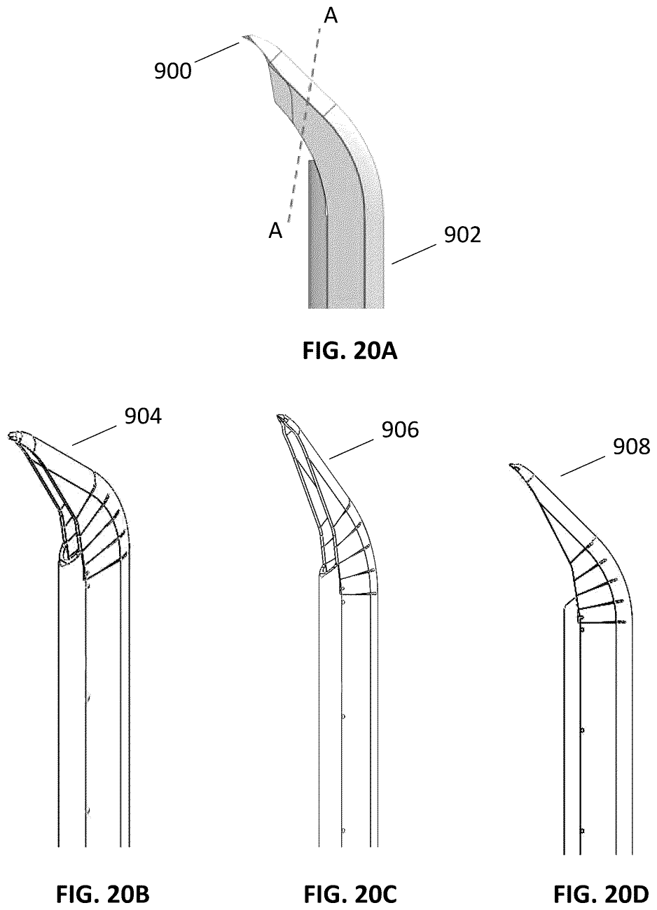

In some variations, as shown in FIG. 20A, the distal tip (900) of the curved cannula (902) may have a sharpness and/or that allows the user to more easily track the distal tip (900) against the plane (depicted as line A-A) of tissue and initiate a puncture within the tissue (providing a tactile advantage to the user). For example, the distal tip (900) may be configured to have a more prominent tip that provides improved tissue interrogation and improved puncture location accuracy. Other exemplary curved cannula tips (904, 906, 908) are shown in FIGS. 20B-20D. In use, as illustrated in FIGS. 21A-21D, a tension element (1000) preloaded within cannula (1002) may be coupled to a pusher (1004). The distal tip of the cannula (1002) may first interrogate tissue and locate a target location for puncture by the cannula tip. The distal tip of the cannula (1000) may then be advanced through tissue (e.g., nasal cartilage 1006) (FIG. 21B) and the distal anchor (1008) in its insertion configuration advanced out of the cannula (1002) using pusher (1004) (FIG. 21C). Upon the application of force by withdrawal of the pusher (1004) and cannula (1002), as shown in FIG. 21D, the distal anchor (1008) may be uncoupled from the pusher and deployed in its deployed configuration by swiveling about a pivot point of the tension element.

The cannula may be made from a polymer. Exemplary polymers include, but are not limited to, acrylic, polycarbonate, polyethylene terephthalate, polyvinyl chloride, polyethylene, polypropylene, and polystyrene. Transparent forms of the aforementioned polymers may be used to make a transparent cannula. In some variations, the cannula may be made from stainless steel or other suitable metals.

Other variations of the cannula may include an internal deflector within the cannula distal end that deflects or angulates the anchor delivery element as it is advanced out of the cannula. The internal deflector may be a flat, rigid surface within the cannula distal end that is angled about 30 degrees to about 70 degrees with respect to the longitudinal axis of the cannula. In other variations, the cannula distal tip may be preformed to have an angle of about 30 degrees to about 70 degrees with respect to the longitudinal axis of the cannula.

The cannula may also have various cross-sectional shapes. For example, the cross-sectional shape of the cannula may be circular, non-circular, semi-circular, or ovular. In some variations where the cannula cross-section is noncircular, the shape may facilitate orientation of the cannula. One or more ports in fluid communication with the lumen may be provided in the cannula for delivery of the tensioning element from the lumen into tissue. The one or more ports may be provided in any suitable location on the cannula, for example, at the distal tip of the cannula or distal side wall of the cannula. The one or more ports may also have any suitable shape. For example, the one or more ports may be circular, semi-circular, or ovular. When a port is provided at the distal tip of the cannula, the port may have a length and a depth. The side profile of the port may also include a curved portion and a flat portion.

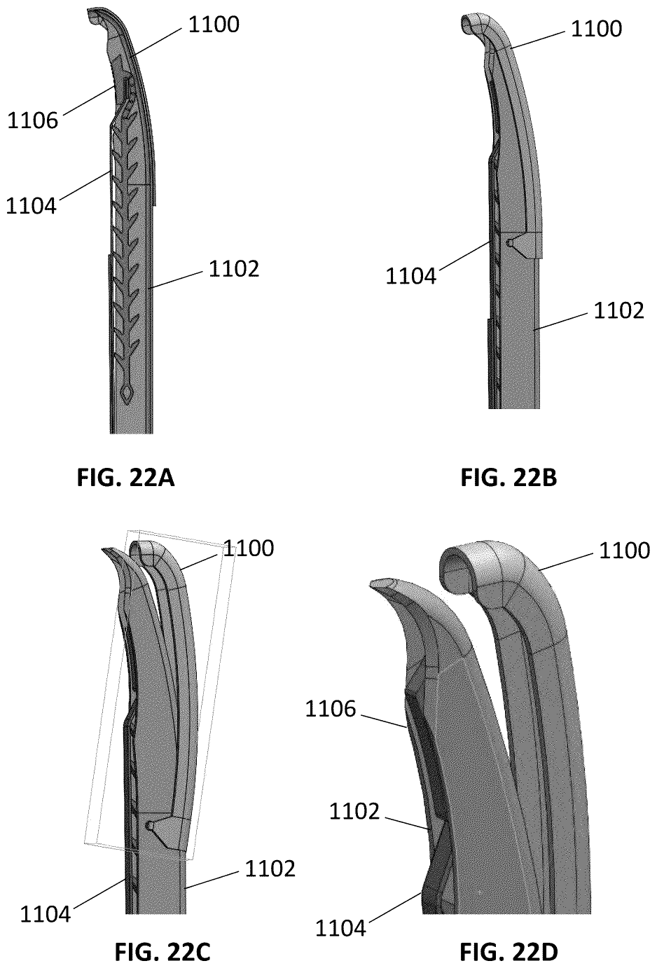

Other variations of the system may include a suction component coupled to the cannula. The back end of the suction component may be configured to connect to standard suction pump. The suction component may be a lumen or hood coupled to the cannula and configured to apply suction to tissue to help with deployment of the cannula distal tip through the tissue. The hood may be configured to protect tissue from damage by the sharp cannula distal tip, e.g., while being advanced through the mucosal tunnel, as well as limit the depth of advancement of the cannula distal tip into tissue. For example, as shown in FIGS. 22A-22D, a suction hood (1100) may be coupled to the curved portion of a cannula (1102). The suction hood (1100) may be actuated by suture or wire that pulls or locks the hood (1100) against the cannula (1102). A spring (not shown) may be used to keep the hood (1100) against the cannula (1102). The hood (1100) may be used to cover the sharp cannula tip while traversing sub-mucosal tissue to prevent damage thereto. The hood may also be used to limit the depth of cannula insertion into tissue. In some instances, the hood (1100) may be configured to deflects back due to apposition with cartilage. For example, a control line may be employed to limit deflection, which limits crossing depth of the cannula tip. In other instances, a pusher wire (1104) may be used to advance the distal anchor (1106) out from the cannula (1102) once the cannula has crossed through a target tissue. More specifically, FIG. 22A shows the suction hood (1100) in a covered position (covering the distal end of the cannula) and the pusher wire (1104) in a sheathed position; FIG. 22B shows the pusher wire (1104) in sheathed position; FIG. 22C shows the suction hood (1100) in a crossing limiter position with the pusher wire (1104) in sheathed position; and FIG. 22D shows a more detailed view of the suction hood (1100) in a crossing limiter position with the pusher wire (1104) in sheathed position.

The shaping of nasal tissue may further be aided by the delivery of one or more fluids to the nasal tissue. In these variations, the tension element may be configured with a fluid delivery mechanism such as a conduit, channel, or other mechanism for suitable delivery of fluid to nasal tissue. This fluid delivery mechanism may allow for the passage of fluid to achieve a therapeutic or physiologic effect. For example, the fluid delivery mechanism may be used to deliver a cold gas or liquid for the purposes of cryotherapy.

### Methods

Methods for reshaping a tissue in a subject are also described herein. The methods may generally include securing a tension element to the tissue, where the tension element comprises an elongate body having a proximal end and a distal end, a pivot point, and a distal anchor at the tension element distal end having a first arm and a second arm. The length of the first arm from the pivot point may be different from the length of the second arm from the pivot point.

In some variations, the distal end of the tension element may be directed through the tissue with an anchor delivery element. The distal anchor may include an anchor body that is configured to swivel (e.g., turn, tilt, flip, rotate, or pivot) about the pivot point. The distal anchor may also have an insertion configuration and a deployed configuration as described above and further herein. After securing the tension element to tissue, a force may be applied to the elongate body to swivel the distal anchor at or about the point from the insertion configuration to the deployed configuration. The force appropriate to manipulate the tissue may then be adjusted by adjusting the tension of the tension element. In some variations, the tension element may also include a plurality of proximal anchors disposed between the distal anchor and the proximal end of the tension element.

In one variation, the method for reshaping a nasal tissue of a subject may include advancing a cannula toward the nasal tissue, and advancing a device including a tension element having an elongate body, a pivot point, and a distal anchor through the cannula. The distal anchor may include a first arm and a second arm, and the first arm length from the pivot point may be different than the second arm length from the pivot point. Additionally, the method may include advancing the distal anchor in an insertion configuration from the cannula through the nasal tissue, and applying a force to the elongate tension element to rotate the distal anchor about a pivot point to transition the distal anchor to a deployed configuration. The device may or may not be preloaded within the cannula. The method may also include advancing one or more proximal anchors through nasal tissue. In some variations, the distal anchor may be advanced using a pusher.

In other variations, the methods may generally include securing a tension element to the tissue, where the tension element comprises an elongate body having a proximal end and a distal end, and a distal anchor at the tension element distal end. In some variations, the distal end of the tension element may be directed through the tissue with an anchor delivery element. The distal anchor may include an anchor body and a pivot point, and an insertion configuration and a deployed configuration. After securing the tension element to tissue, a force may be applied to the elongate body to swivel the distal anchor at the pivot point from the insertion configuration to the deployed configuration. The force appropriate to manipulate the tissue may then be adjusted by adjusting the tension of the tension element.

The proximal and distal ends of the elongate body of the tension element may be secured to the same tissue. Alternatively, the proximal and distal ends of the elongate body may be secured to different tissues. The tissue may be a nasal tissue, a throat tissue, or an ear tissue. Exemplary nasal tissues include without limitation, nasal septal cartilage, lateral nasal cartilage, major alar cartilage, minor alar cartilage, alar fibrofatty tissue, nasal bone, or a nasal turbinate. Exemplary throat tissues include without limitation, the uvula, soft palate, and tonsils. Non-limiting examples of ear tissues include cartilage of the helix, anti-helix, tragus, anti-tragus, superior crus, fossa triangularis, concha, and connective tissue of the earlobe.

The methods described herein may be used to treat various conditions and manipulate various tissues. For example, the manipulation of tissue by the tension elements may be used to treat nasal septal deviation, lateral nasal valve collapse, and other causes of nasal airway obstruction. Additionally, the manipulation of tissue may be used to medialize a middle turbinate, compresses or lateralize the inferior turbinate, or reapproximate nasal mucosa. Furthermore, the manipulation of tissue by the tension elements may alter the shape of various tissues. For example, the shape of a nasal tissue, a throat tissue, or an ear tissue may be altered. When the tissue is a nasal tissue, the tissue may include lateral cartilage, alar cartilage, columella, or a combination thereof. Additionally, the manipulation of tissue may be used to increase the stiffness or rigidity of a nasal tissue, a throat tissue, or an ear tissue. In some variations, the tension elements may be employed in minimally invasive face lift procedures.

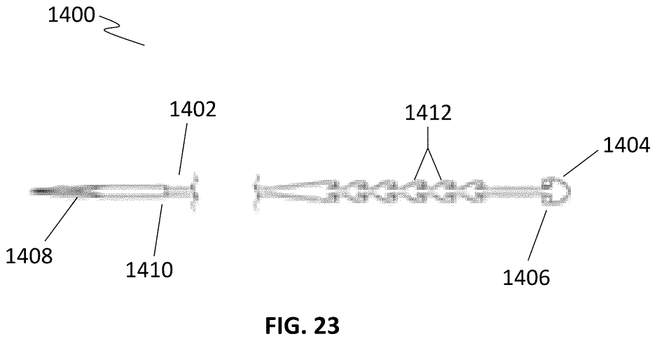

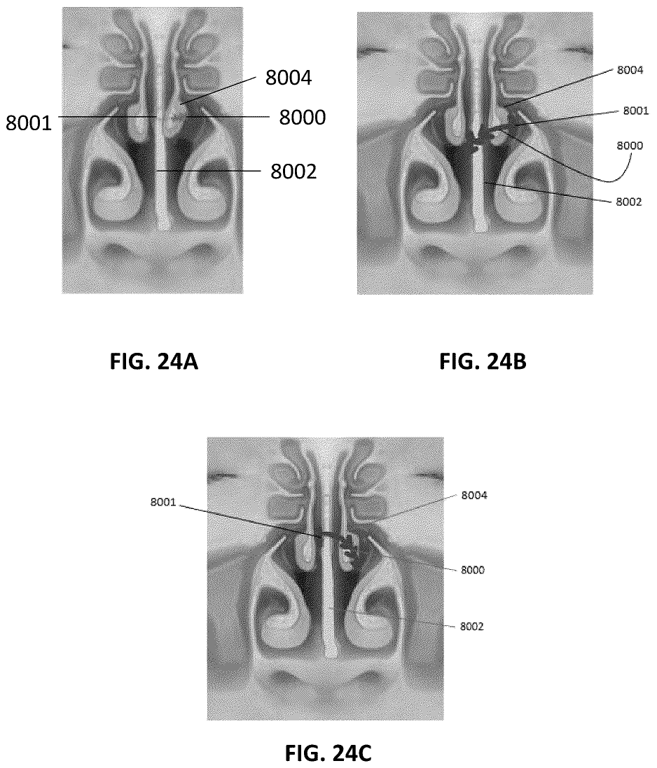

In some patients, one or more middle turbinates may already be destabilized (e.g., floppy), or middle turbinate destabilization may result from sinus surgery. Sinus surgery may also result in adhesions forming between middle turbinate mucosa and lateral wall mucosa, which in turn may block off the osteomeatal complex. Middle turbinate lateralization may be the most common complication after FESS (Functional Endoscopic Sinus Surgery) and may occur in up to approximately 45% of sinus surgeries where the middle turbinate is not stabilized. The devices described herein may be used to medialize and stabilize the middle turbinates, preventing lateral wall adhesions during healing, as shown in FIGS. 24A to 24C. In some instances, the tension element shown in FIG. 23 may be used to medialize or lateralize one or more turbinates. Referring to FIG. 23, the tension element (1400) may include an elongate body (1402), a distal anchor (1404) at a distal end (1406) of the elongate body (1402), and a detachable needle (1408) at a proximal end (1410) of the elongate body (1402). The proximal needle may be removable attached to the tension element by swaging or crimping. The tension element may also include a plurality of proximal anchors (1412) on the elongate body (1402). The number of proximal anchors may be less than that used for reshaping a nasal septum, e.g., four or five proximal anchors, and the distal-most proximal anchor may be spaced from the distal anchor by about 1.0 mm to about 6.0 mm, including all values and sub-ranges therein. For example, the distal-most proximal anchor may be spaced from the distal anchor by about 1.0 mm, about 2.0 mm, about 3.0 mm, about 4.0 mm, about 5.0 mm, or about 6.0 mm.

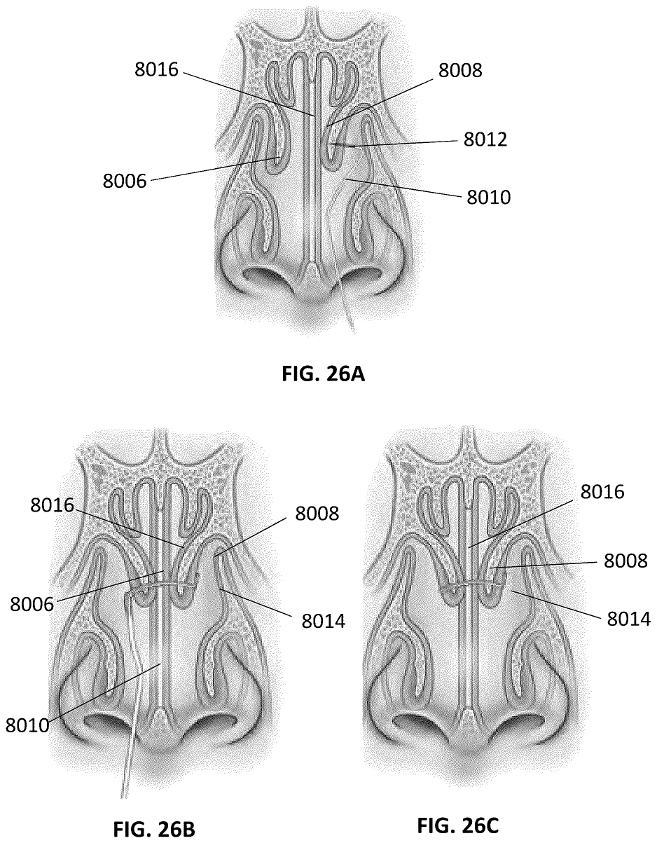

Referring to FIG. 24A, the method may include inserting a distal anchor (8001) of a tension element (8000) into nasal septal cartilage (8002), wrapping the tension element around the middle turbinate (8004), applying a force to the tension element to pull the middle turbinate (8004) medially toward the nasal septal cartilage (8002), and maintaining the medial position of the middle turbinate by securing the proximal end of the tension element (8000) to the nasal septal cartilage (8002). Securing the tension element proximal end may be accomplished by any suitable method. In one variation, suturing the proximal end to the nasal septal cartilage secures the tension element such that it maintains the force needed to medialize the middle turbinate. Alternatively, and as shown in FIG. 24B, the method for medializing the middle turbinate may involve inserting a distal anchor (8001) of a tension element (8000) into a middle turbinate (8004), applying a force to the tension element (8000) to pull the middle turbinate (8004) medially toward the nasal septal cartilage (8002), and maintaining the medial position of the middle turbinate (8004) by securing the proximal end of the tension element (8000) to the nasal septal cartilage (8002). Securing the tension element proximal end may be accomplished by any suitable method. As previously mentioned, suturing the proximal end to the nasal septal cartilage secures the tension element such that it maintains the force needed to medialize the middle turbinate. In FIGS. 26A to 26C, an alternative method to medialize turbinates is shown where a proximal needle (8012) is first inserted through both a first middle turbinate (8008) and a second middle turbinate (8006) (FIGS. 26A and 26B), a distal anchor (8014) secured against the first middle turbinate (8008), applying a force to the tension element (8010) to pull the first middle turbinate (8004) medially toward the nasal septal cartilage (8016), and maintaining the medial position of the first and second middle turbinates (8008, 8006) by securing a proximal end of the tension element (8010) to the second middle turbinate (8006). Securing the tension element proximal end may be accomplished by any suitable method, e.g., suturing.

In yet a further variation, as shown in FIG. 24C, the method for medializing the middle turbinate may include inserting a distal anchor (8001) of a tension element (8000) into nasal septal cartilage (8002), passing the proximal end of the tension element (8000) through the middle turbinate (8004), and securing the middle turbinate (8004) in a medialized position by securing the proximal end of the tension element (8000) to the middle turbinate (8004).

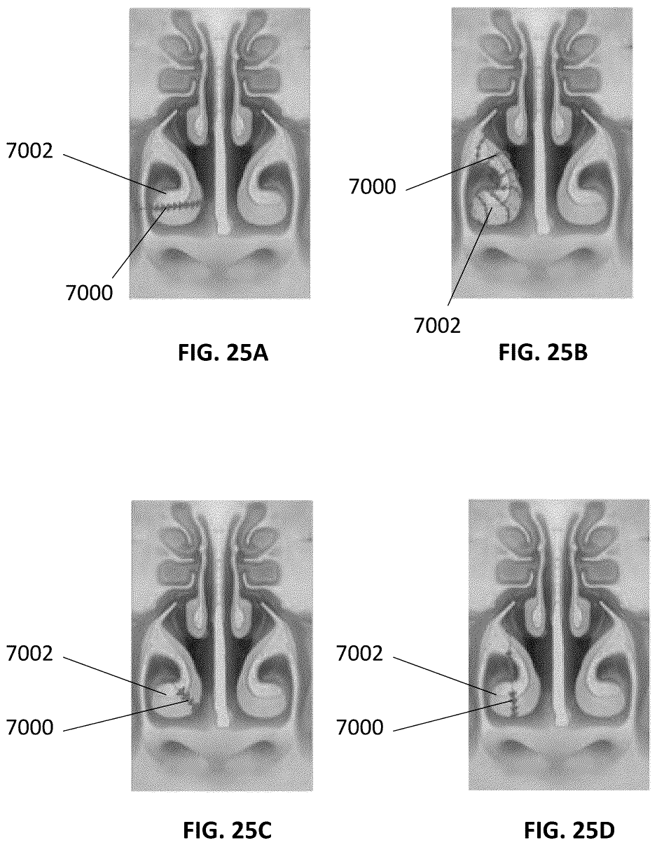

As shown in FIGS. 25A-25D, methods for treating inferior turbinate hypertrophy may include wrapping a tension element (7000) around the inferior turbinate (7002). A single turn may be used to wrap the tension element (7000) around the outer edges of the inferior turbinate (7002) to reduce its diameter (FIG. 25A). In another variation, multiple turns may be used to wrap a tension element around the entire inferior turbinate to compress and reduce the caliber of the inferior turbinate (FIG. 25B). Alternatively, a single turn may be used to wrap the tension element (7000) so that it is mobilized laterally (FIG. 25C) or superiorly (FIG. 25D).

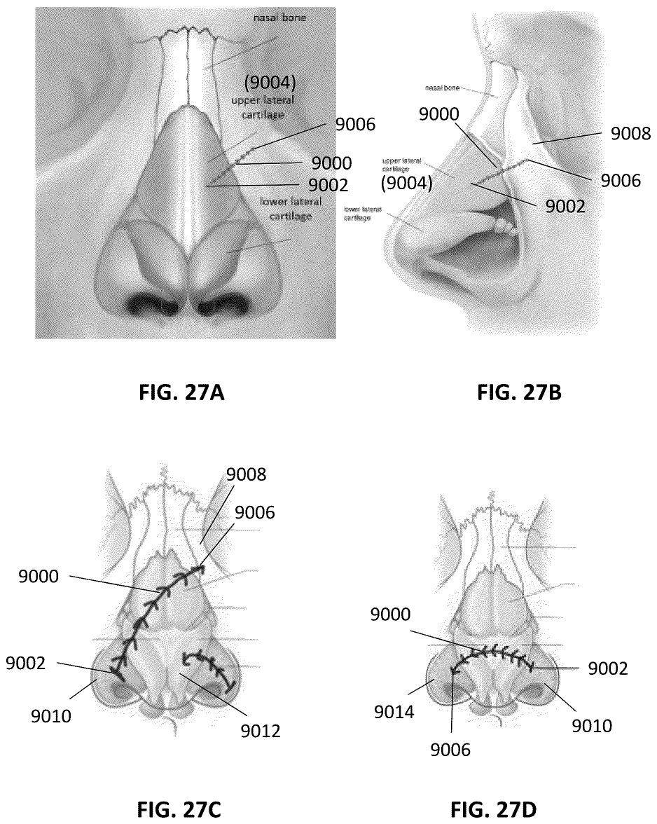

Lateral nasal valve collapse may also be treated with the tension elements described herein. As shown in FIGS. 27A-27D, a tension element (9000) may be anchored between two nasal tissues and a tension force applied therebetween to increase the patency of the nasal valve. For example, in FIGS. 27A and 27B, the distal end (9002) of tension element (9000) may be anchored in the upper lateral cartilage (9004) and the proximal end (9006) of the tension element (9000) secured in the maxillary bone (9008). As shown in FIG. 27C, the distal end (9002) of tension element (9000) may be anchored in fibrofatty tissue (9010), and the proximal end (9006) secured in the maxillary bone (9008) or a lower lateral cartilage (9012). Alternatively, as shown in FIG. 27D, the distal end (9002) of a tension element (9000) may be anchored in fibrofatty tissue (9010) and the proximal end (9006) secured to fibrofatty tissue on the opposing side (9014) of the nose.

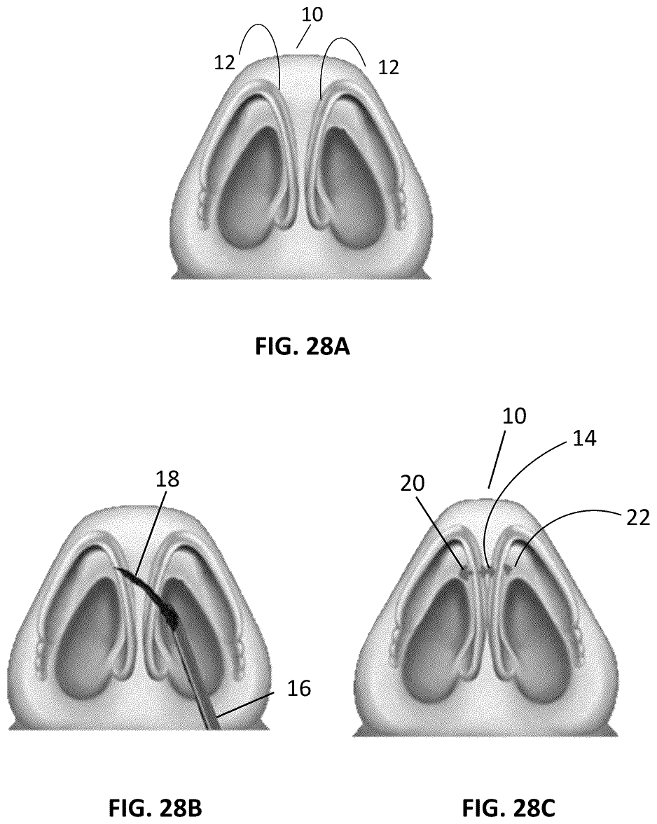

Nasal tip reshaping may further be accomplished with the tension elements described herein. As shown in FIGS. 28A-28C, the shape of nasal tip (10) may be altered by pulling lateral cartilages (12) medially. To this end, a tension element (14) coupled to an anchor delivery element (18) may be deployed from a delivery cannula (16) such that the distal anchor (20) of the tension element (14) is secured to one nasal cartilage (12) and the proximal end (22) of the tension element (14) is secured to the other nasal cartilage (12). A tension force provided between the distal anchor and the tension element proximal end may draw the lateral cartilages together to thereby alter the shape of the nasal tip (10).

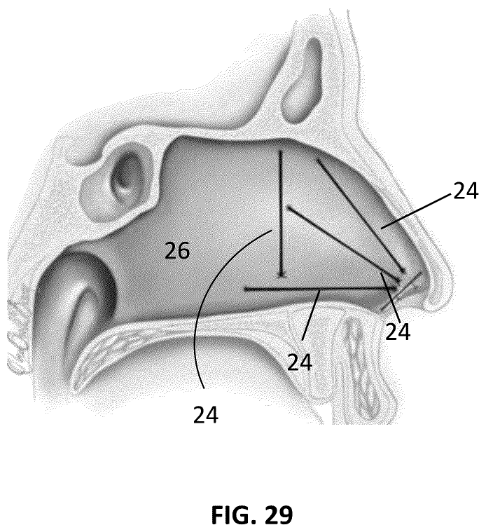

In some variations, the method may include reapproximating nasal mucosa to prevent the formation of nasal hematoma, for example, after nasal septoplasty. Referring to FIG. 29, a tension element (24) may be placed on the surface of the nasal mucosa (26) in one or more locations shown in the figure to tack down the mucosa and prevent any negative spaces from forming that could fill with blood.

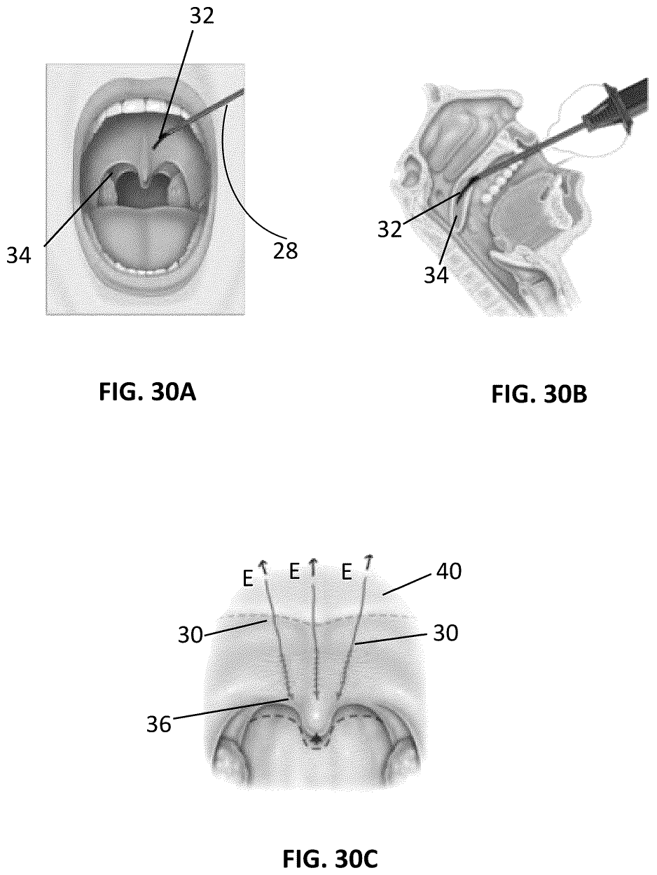

In other variations, the method may include placing one or more tension elements in throat tissue to treat obstructive sleep apnea. As shown in FIGS. 30A-30C, the method may include advancing a delivery cannula (28) carrying a tension element (30) coupled to an anchor delivery element (32) to the throat of a subject, inserting the anchor delivery element (32) into the uvula (34), setting the distal anchor (36) of the tension element (30) into tissue of the uvula, applying a tension force on the tension element (30) and distal anchor (36) in the direction of arrow E to lift the uvula, and securing the proximal end (38) of the tension element (30) in tissue of the soft palate (40) to maintain the tension force on the uvula (34). One or a plurality of tension elements may be employed to lift the uvula and treat obstructive sleep apnea.

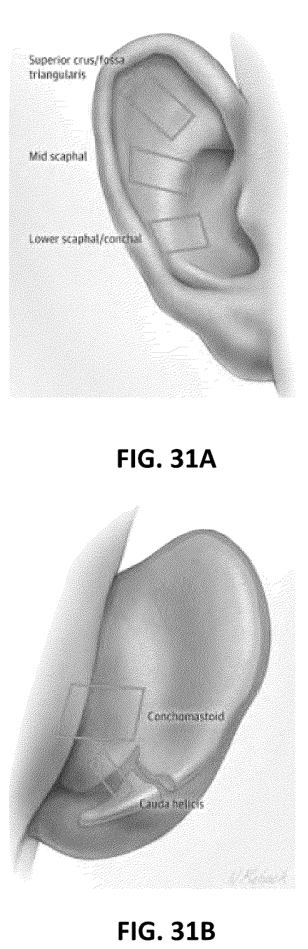

The reshaping of ear tissues may also be accomplished with the devices described herein. In some variations, reshaping is used to treat a poorly defined antihelix, for example, by creating or increasing the antihelical fold. In other cases, reshaping may be used to correct enlarged conchal cartilage. As shown in FIGS. 31A and 31B, the method to more clearly define the antihelix may include placing a tension element in one or more of the locations shown in the figures. For example, the tension element may be placed at the superior crus or fossa triangularis, the midportion of the scapha, the lower portion of the scapha or concha, or the conchomastoid and cauda helicis regions of the posterior ear.

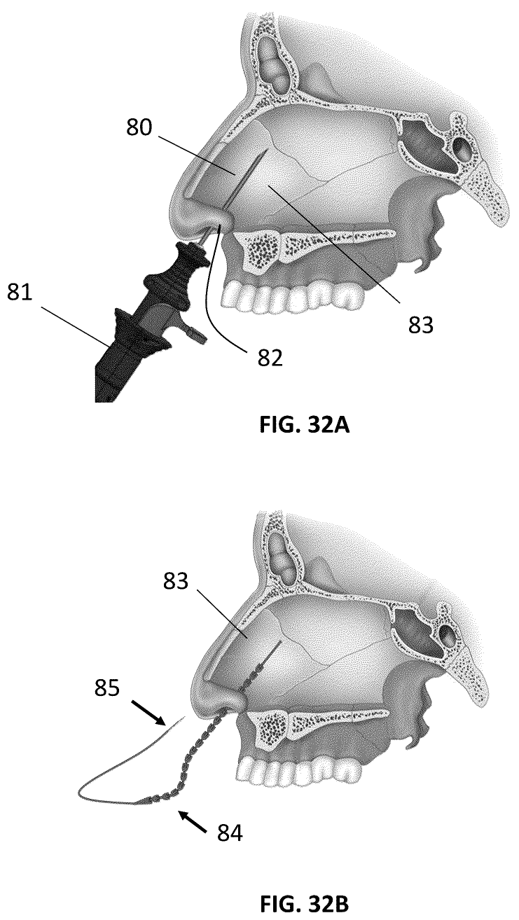

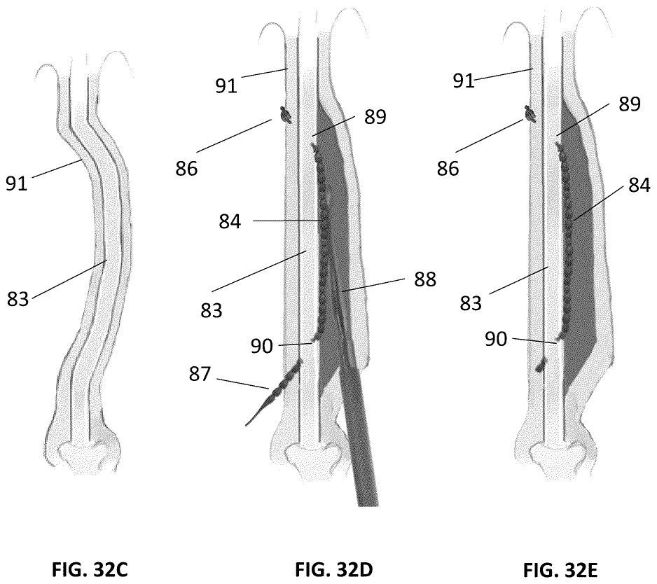

The methods may be used to shape the nasal septum, as shown in FIGS. 32A to 32E. A deviated nasal septum (83) is illustrated in FIG. 32C. Referring to FIGS. 32A to 32E, the method for shaping the deviated nasal septum (83) may include inserting a cannula (80) of a delivery device (81) into a nostril (82) and through the nasal septum (83) at a first location (89), securing a distal anchor (86) into the nasal cartilage (91), and deploying a tension element (84) from the delivery cannula (80). The proximal end (87) of the tension element (84) may then be passed through the nasal septum (83) at a second location (90). A force may be applied to the proximal end (87) of the tension element (84), which may create a medial force against the deviated nasal septum (83) to shape the tissue by straightening the tissue, as shown in FIG. 32D. Additional medial force may be applied by a surgeon using an instrument (88) to straighten the nasal septum (83). Once the desired amount of shaping is achieved, the force on the tension element (84) may be held by securing the proximal end (87) of the tension element (84) to the nasal septum (83) or nasal cartilage (91) near the second location (90).

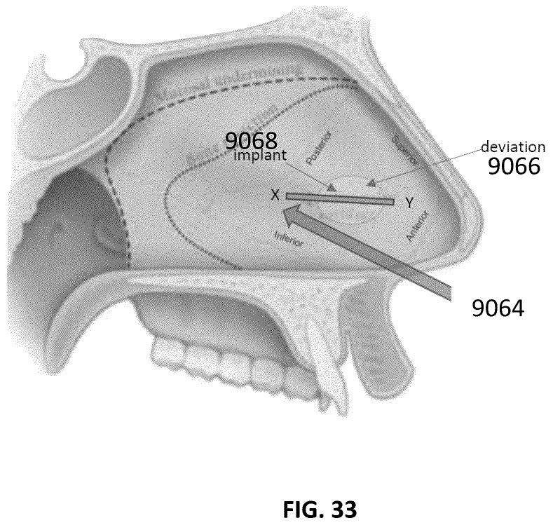

The cannula may be advanced through an access site in submucosal tissue on a first side of the nasal septum and through the nasal septum to a second side of the nasal septum. In some instances, the cannula may access a location anterior to the deviation and create a submucosal tunnel beneath the deviation. A distal anchor of a tension element may then be secured into nasal cartilage on the second side of the nasal septum (e.g., posterior to the deviation), where the distal anchor comprises a pivot point and has an insertion configuration and a deployed configuration. The method may include securing the distal anchor by applying a force to the elongate tension element to swivel the distal anchor at the pivot point from the insertion configuration to the deployed configuration. In some variations, the method may further include passing a proximal end of the elongate tension element back through the access site in submucosal tissue to the second side of the nasal septum, tensioning the elongate tension element to a tensioned state, and securing the proximal end of the elongate tension element in its tensioned state to tissue on the second side of the nasal septum. For example, referring to FIG. 33, a submucosal tunnel (9064) may be created with a delivery device cannula (not shown) below deviation (9066) for placement of a distal anchor (X) and a proximal anchor (Y) of a tension element (9068) to reshape the deviated nasal septum. The tension element in its tensioned state may apply a force ranging from about 1.0 Newton to about 70 Newtons (including all values and sub-ranges therein) to the deviated nasal septal tissues.

The force applied to manipulate or shape a tissue may range from about 4.0 Newtons to about 70 Newtons, including all values and sub-ranges therein. The force may be generated by pulling on the free proximal end of the tension element after the distal anchor has been fixed to the target tissue. For example, the tension force may be about 4.0 Newtons, about 5.0 Newtons, about 10 Newtons, about 15 Newtons, about 20 Newtons, about 25 Newtons, about 30 Newtons, about 35 Newtons, about 40 Newtons, about 45 Newtons, about 50 Newtons, about 55 Newtons, about 60 Newtons, about 65 Newtons, or about 70 Newtons. Tensile strength of the tension element may range from about 100 Mpa to about 300 Mpa, including all values and sub-ranges therein. For example, the tensile strength may be about 100 Mpa, about 110 Mpa, about 120 Mpa, about 130 Mpa, about 140 Mpa, about 150 Mpa, about 155 Mpa, about 160 Mpa, about 165 Mpa, about 170 Mpa, about 175 Mpa, about 180 Mpa, about 185 Mpa, about 190 Mpa, about 195 Mpa, about 200 Mpa, about 210 Mpa, about 220 Mpa, about 230 Mpa, about 240 Mpa, about 250 Mpa, about 260 Mpa, about 270 Mpa, about 280 Mpa, about 290 Mpa, or about 300 Mpa. In some instances, the tensile strength of the tension element may be at least about 150 Mpa. In other instances, the tensile strength of the tension element may be at least about 300 Mpa. The applied force may decrease over time as the tension element biodegrades. In general, the tension element biodegrades over a period of about one months to about twelve months. For example, the tension element may biodegrade over a period of at least about one month, about two months, about three months, about four months, about five months, about six months, about seven months, about eight months, about nine months, about ten months, about eleven months, or about twelve months. In one variation, the tension element may degrade over a period ranging from about four months to about nine months.

The methods described herein may be used for treatment of nasal airway obstruction; treatment of a deviated nasal septum; straightening of a nasal septum; treatment of a thickened, deformed, or dislocated nasal septum; repair of nasal septal fracture; alteration of the shape of the nasal septum; treatment of nasal septal spurs or nasal bone spurs; alteration of the shape of the internal or external shape of the nose; treatment or alteration of structural deformity of a nasal cartilage other than the nasal septum; treatment of internal nasal valve collapse; or treatment of turbinate hypertrophy. The method may also be employed to treat or alleviate sleep apnea, nasal snoring, or may be configured for any other suitable alteration of nasal tissue or any combination of tissues. In other variations, the methods described herein may be used to buttress or aid in approximating broken nose fragments.

### Additional Exemplary Devices, Delivery Systems, and Methods

### Reshaping of Nasal Tissue

When nasal tissue, e.g., nasal septal tissue, is to be reshaped (repositioned, compressed, approximated, etc.), the device may include a tension element comprising an elongate body having a proximal end, a distal end, a pivot point at the distal end, a distal anchor distal to pivot point, a plurality of proximal anchors disposed between the distal anchor and the proximal end of the elongate body, and a retainer disposed on the elongate body between the distal anchor and a first proximal anchor of the plurality of proximal anchors. The retainer may be configured to releasably attach to a portion of a delivery system, e.g., a pusher of the delivery system. The distal anchor may include a first arm and a second arm, where the length of each of arm is different from the pivot point. However, in some instances, the first and second arms may be the same length. The distal anchor may have an insertion configuration that allows for easier advancement through tissue, and a deployed configuration that helps to firmly fix the distal anchor in place and prevent dislodgment (e.g., pull back through the incision/access site in the nasal tissue) upon the application of force to the tension element.

Similar to that described above, the distal anchor may be configured to rotate about the pivot point from the insertion configuration to the deployed configuration upon the application of force to the elongate body. The end of one or more of the first and second arms may have a shape configured to initiate pivoting of the distal anchor upon the application of force to the elongate body. The shape may comprise one or more of a bevel, taper, or chamfer. In some variations, the distal anchor may have a triangular profile. Additionally, the distal anchor in the deployed configuration may generally have a larger surface area for opposing tissue than the distal anchor in the insertion configuration.

The plurality of proximal anchors may comprise one or more of a barb, hook, spike, tooth, leaf, nub, and protrusion. A distal end of each of the plurality of proximal anchors may be beveled, tapered, sharp, curved, or a combination thereof. In some variations, the plurality of proximal anchors may be symmetrically spaced apart along a length of the elongate body. In other variations, the plurality of proximal anchors may be asymmetrically spaced apart along a length of the elongate body. The plurality of proximal anchors may be staggered (offset) along the length of the elongate body. A first proximal anchor (e.g., the most distal anchor of the plurality of proximal anchors) of the plurality of proximal anchors may be spaced between about 2.0 mm to about 8.0 mm (including all values and sub-ranges therein) from the distal anchor. For example, the first proximal anchor of the plurality of proximal anchors may be spaced from the distal anchor a distance of about 2.0 mm, about 2.5 mm, about 3.0 mm, about 3.5 mm, about 4.0 mm, about 4.5 mm, about 5.0 mm, about 5.5 mm, about 6.0 mm, about 6.5 mm, about 7.0 mm, about 7.5 mm, or about 8.0 mm. In one variation, the first proximal anchor of the plurality of proximal anchors may be spaced from the distal anchor a distance of about 7.6 mm. The plurality of proximal anchors may be provided on a region of the elongate body having a length ranging from about 1.5 cm to about 6.5 cm, including all values and ranges therein. For example, the plurality of proximal anchors may be provided on a length of the elongate body of about 1.5 cm, about 2.0 cm, about 2.5 cm, about 3.0 cm, about 3.5 cm, about 4.0 cm, about 4.5 cm, about 5.0 cm, about 5.5 cm, about 6.0 cm, or about 6.5 cm. In some variations, the length of the elongate body including the proximal anchors is about 2.5 cm.

The elongate body of the tension element may be flexible, and may be straight or have at least a portion with an undulating or wave-like shape. In some variations, at least the portion of the elongate body upon which the proximal anchors are disposed has an undulating or wave-like shape. The proximal end of the elongate body may comprise an eyelet for coupling to a suture. In some variations, a needle may be releasably coupled to the proximal end of the elongate body.

As described above, the tension element may comprise a biodegradable polymer. The biodegradable polymer may be selected from the group consisting of LPLA (Poly(L-lactide)), DLPLA (Poly(DL-lactide)), LDLPLA (Poly(DL-lactide-co-L-lactide)), LPLA-HA (Poly(L-lactide) with hydroxylapatite), PGA (Poly(glycolide)), PGA-TMC (Poly(glycolide-co-trimethylene carbonate) or polyglyconate), PDO (Poly(dioxanone)), LPLG (Poly(L-lactide-co-glycolide)), DLPLG (Poly(DL-lactide-co-glycolide), and copolymers and blends thereof. In one variation, the tension element may comprise PDO (Poly(dioxanone)). In other variations, the tension element may comprise a non-biodegradable polymer or metal, as previously described herein.

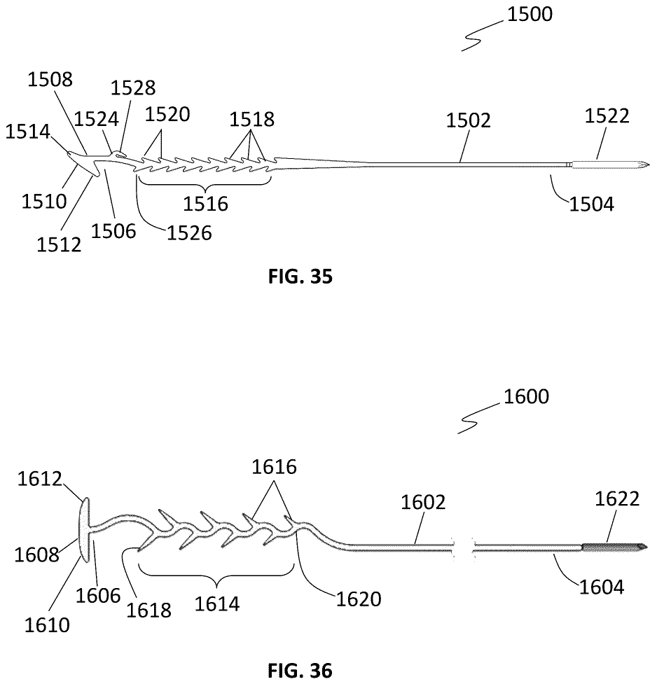

An exemplary device for reshaping (repositioning, compressing, approximating) nasal tissue, e.g., nasal septal tissue, is shown in FIG. 35. In FIG. 35, the tension element (1500), which is shown in an insertion configuration, may include an elongate body (1502) having a proximal end (1504), a distal end (1506), and a pivot point (1508) at the distal end (1506). The tension element (1500) may also include a distal anchor (1510) distal to the pivot point (1508) that comprises a first arm (1512) and a second arm (1514). The first and second arms (1512, 1514) have a length measured from the pivot point, and the length of the first arm (1512) from the pivot point (1508) may be different than the length of the second arm (1514) from the pivot point (1508). However, as previously mentioned, in some instances the first and second arm lengths may be the same. In FIG. 35, the length of the first arm (1512) is shorter than the length of the second arm (1514) from the pivot point (1508), and each of the first and second arms (1512, 1514) is also triangular in profile. Compared to a T-anchor having arms with a straight profile, the triangular arm profile may improve resistance to deformation upon application of force to the tension element. The asymmetric geometry of the distal anchor (1510) may also help to facilitate transitioning of the distal anchor (1510) from the insertion configuration to the deployed configuration and increase retention force by causing the distal anchor (1508) to sit perpendicular to the incision made by the delivery device.

The tension element (1500) may also include a plurality of proximal anchors (1516). The plurality of proximal anchors (1516) may comprise teeth (1518) that are staggered/offset from each other along a length of the elongate body (1502). The staggered/offset pattern of the proximal anchors may improve anchoring of the tension element and cause the tension element to oscillate as it is pulled through tissue, thereby minimizing tissue distension. The region of the elongate body along which the teeth (1518) are disposed may have a length ranging from about 1.5 cm to about 6.5 cm, e.g., about 2.5 cm. The tips (1520) of the teeth (1518) may generally point in the distal direction (direction of the distal anchor), but may point in other directions. Additionally, a needle (1522) may be releasably coupled to the proximal end (1504) of the tension element (1502). The proximal needle (1522) may be used to place or manipulate the proximal end (1504) of the elongate body (1502) through or around tissue.

The tension element (1500) may further include a retainer (1524) between the distal anchor (1510) and a first proximal anchor (1526) of the plurality of proximal anchors (1516). As further described below, the retainer (1524) may be configured to releasably attach to a portion of a delivery system, e.g., a pusher of the delivery system. The retainer (1524) may comprise a clasp or rententive arm (1528). The clasp or retentive arm (1528) may be configured to extend proximally towards the proximal end (1504) of the tension element (1500) such that clasp or retentive arm (1528) may attach to a component (e.g., pusher) of the delivery system during advancement of the component through tissue, and detach from the component (e.g., pusher) during retraction of the component back into the delivery system and/or withdrawal of the delivery system from the site of tension element implantation. Tension element (1500) may be useful when nasal septal tissue is being reshaped to treat a deviated nasal septum.

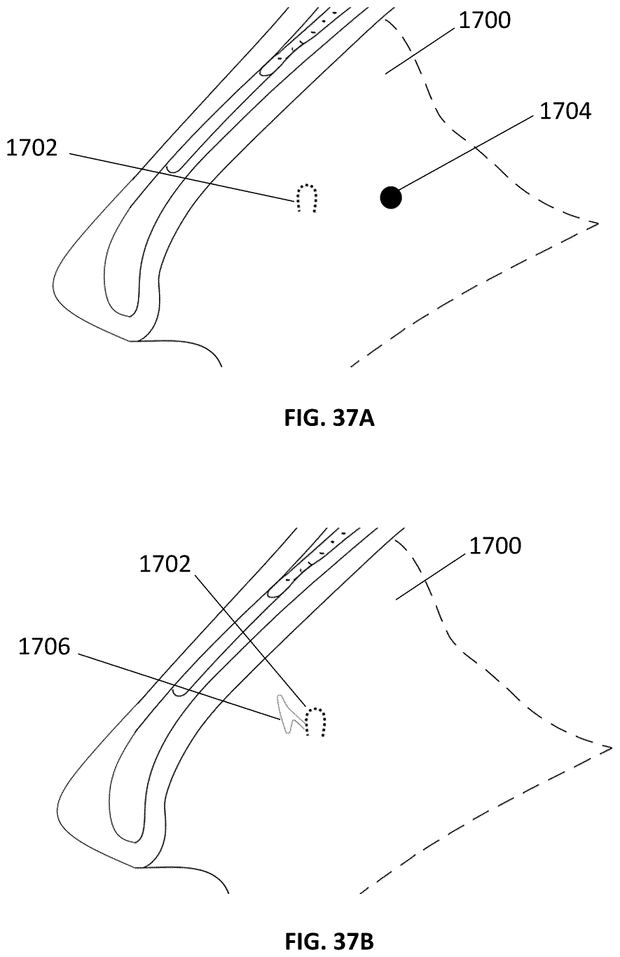

Delivery systems useful in deploying tension element (1500) (and other reshaping devices described herein) may be configured to create an arc-shaped incision (e.g. a U-shaped incision or an upside down U-shaped incision depending on the orientation of the delivery system cannula) in nasal tissue, e.g., the nasal septum. For example, referring to FIGS. 37A and 37B, the delivery systems may be configured to form an arc-shaped incision (1702) in the nasal septum (1700) instead of a puncture hole (1704) generally made by cannulas of conventional delivery systems, and thus provides an improvement over these systems. This is because the arc-shaped incision (1702) may help prevent dislodgement of a distal anchor (1706) by providing a smaller opening than a puncture hole for the distal anchor (1706) to pass back through when a force is applied to the tension element (not shown).

In general, the delivery systems may include a handle and a cannula extending from the handle. The handle may comprise a translational actuator configured to advance a reshaping device through the distal portion of the cannula. In some variations, the handle may include a lock configured to permit translational movement of the actuator when depressed. The delivery devices may be preloaded with any of the reshaping devices described herein, e.g., the device/tension element shown in FIG. 35. However, in some variations, the delivery devices are not preloaded, and the reshaping devices are loaded into the delivery device just before or during the nasal procedure.

The cannula of the delivery systems may include a proximal end and a distal portion, where the distal portion may comprise a curved upper surface, a sharp distal tip, and a lower surface comprising a first cutting edge and a second cutting edge, where the first and second cutting edges and the sharp distal tip are configured to create a shaped path, e.g., an arc-shaped incision in a target nasal tissue, e.g., the nasal septum. In other instances, the incision may create an angular path like a letter ‘V’ or part of a rectangle ‘|_|’, or a combination of a smooth and angular paths. The curved upper surface may have an angle of curvature ranging from about 15 degrees to about 60 degrees, including all values and sub-ranges therein. For example, the angle of curvature may be about 15 degrees, about 20 degrees, about 25 degrees, about 30 degrees, about 35 degrees, about 40 degrees, about 45 degrees, about 50 degrees, about 55 degrees, or about 60 degrees. In some variations, the angle of curvature is about 45 degrees. The curved upper surface may be a non-cutting surface. After deployment from the delivery device, the elongate body of the tension element may be seated in the incision, and the distal anchor implanted in tissue behind the tissue flap created by the arc-shaped incision, as illustrated in FIG. 37B. The curved surface of the cannula may have an angle of curvature configured to decrease the amount of force applied to advance a distal tip of the cannula through tissue (providing a mechanical advantage to the user).

In some variations, the cannula of the delivery system may be made from a polymer. Exemplary polymers include, but are not limited to, acrylic, polycarbonate, polyethylene terephthalate, polyvinyl chloride, polyethylene, polypropylene, and polystyrene. Transparent forms of the aforementioned polymers may be used to make a transparent cannula. In other variations, the cannula may be made from stainless steel or other suitable metals. The cannula may include markings thereon to help facilitate tension element placement. When made from a metal, the markings may be laser markings.

The cannula length may range from about 50 mm to about 70 mm, including all values and sub-ranges therein. For example, the length of the cannula may be about 50 mm, about 55 mm, about 60 mm, about 65 mm, or about 70 mm. The cannula may also have various cross-sectional shapes. For example, the cross-sectional shape of the cannula may be circular, non-circular (e.g., square, rectangular), semi-circular, or ovular. In some variations where the cannula cross-section is noncircular, the shape may facilitate orientation of the cannula.

The delivery systems generally also include a pusher disposed within the cannula, where the pusher may comprise a proximal end, a distal end, and a coupler at the distal end configured for releasable attachment to a retainer of a reshaping device, e.g., the retainer (1524) shown in FIG. 35. The pusher of the system may be flexible, and may include a plurality of slits along its length. For example, the pusher may comprise a hypotube including a plurality of laser cuttings (laser cut hypotube). The plurality of slits/cuts may be symmetrically or asymmetrically spaced apart along the length of the pusher. The plurality of slits/cuts along the length of the pusher may assist with advancement of the pusher through the curve of the cannula. The diameter of the pusher may be any suitable diameter that allows the pusher to be concentrically disposed within, and slidable through, the cannula. The length of the pusher may range from about 10 cm to about 20 cm, including all values and sub-ranges therein. For example, the length of the pusher may be about 10 cm, about 11 cm, about 12 cm, about 13 cm, about 14 cm, about 15 cm, about 16 cm, about 17 cm, about 18 cm, about 19 cm, or about 20 cm. In one variation, the length of the pusher is about 13 cm.

Additionally, the distal portion of the cannula may comprise a stop region configured to limit a puncture depth of the sharp distal tip. The stop region may be configured to limit the length of the distal portion (including the distal tip) passing through the nasal septum to between about 4.0 mm to about 15 mm, including all values and sub-ranges therein. For example, the stop region may limit the length of the distal portion (including the distal tip) passing through the nasal septum to about 4.0 mm, about 5.0 mm, about 6.0 mm, about 7.0 mm, about 8.0 mm, about 9.0 mm, about 10 mm, about 11 mm, about 12 mm, about 13 mm, about 14 mm, or about 15 mm. In one variation, the stop region may limit the length of the distal portion passing through the nasal septum to about 9.0 mm. In some variations, the delivery system may further include a depth guard configured for removable attachment to the cannula, which may also help limit the length of the distal portion passing through the nasal septum by providing a barrier to further advancement of the cannula distal tip. The amount of force typically needed to puncture through the nasal septum is about 1.0 lbf to about 2.0 lbf (about 4.0 Newtons to about 9.0 Newtons). When a depth guard is provided on the cannula, the amount of force that may be required to puncture past the depth guard may be about 9.0 lbf (about 40 Newtons). The depth guard may be configured as a sleeve including various cutouts for passage of the distal portion of the cannula and ease of attachment and/or removal from the cannula. The depth guard may have a length (from proximal end to distal end) ranging from about 50 mm to about 60 mm, including all values and sub-ranges therein. For example, the depth guard may have a length of about 50 mm, about 51 mm, about 52 mm, about 53 mm, about 54 mm, about 55 mm, about 56 mm, about 57 mm, about 58 mm, about 59 mm, or about 60 mm. The width of the depth guard may range from about 2.0 mm to about 3.0 mm, including all values and sub-ranges therein. For example, the width may be about 2.0 mm, about 2.1 mm, about 2.2 mm, about 2.3 mm, about 2.4 mm, about 2.5 mm, about 2.6 mm, about 2.7 mm, about 2.8 mm, about 2.9 mm, or about 3.0 mm. In one variation, the width of the depth guard may be about 2.2 mm. The height of the depth guard may range from about 5.0 mm to about 7 mm, including all values and sub-ranges therein. For example, the height may be about 5.0 mm, about 5.1 mm, about 5.2 mm, about 5.3 mm, about 5.4 mm, about 5.5 mm, about 5.6 mm, about 5.7 mm, about 5.8 mm, about 5.9 mm, about 6.0 mm, about 6.1 mm, about 6.2 mm, about 6.3 mm, about 6.4 mm, about 6.5 mm, about 6.6 mm, about 6.7 mm, about 6.8 mm, about 6.9 mm, or about 7.0 mm. In one variation, the height of the depth guard may be about 6.6 mm.

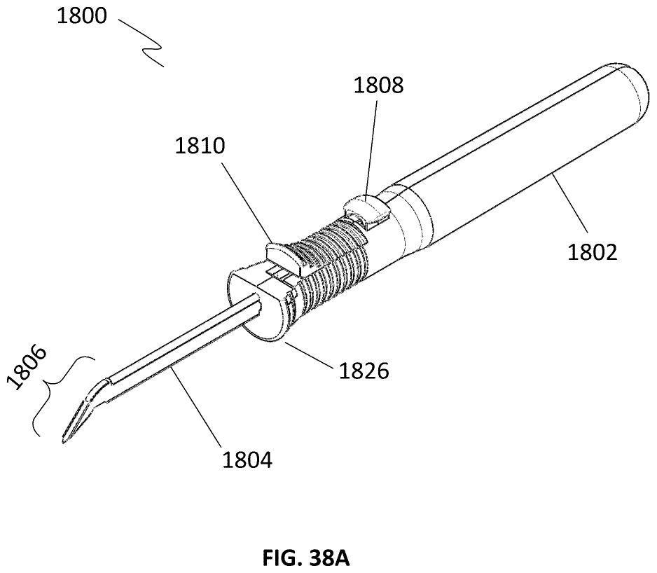

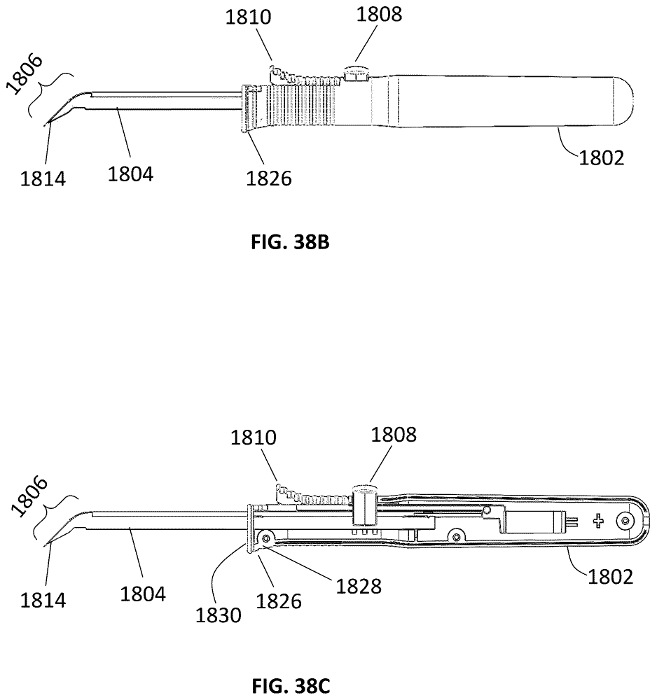

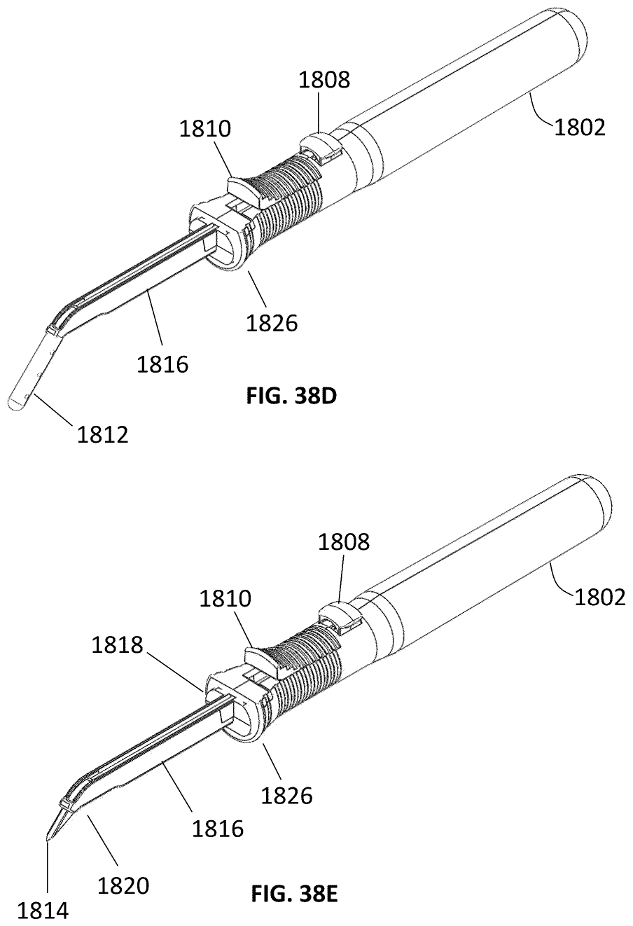

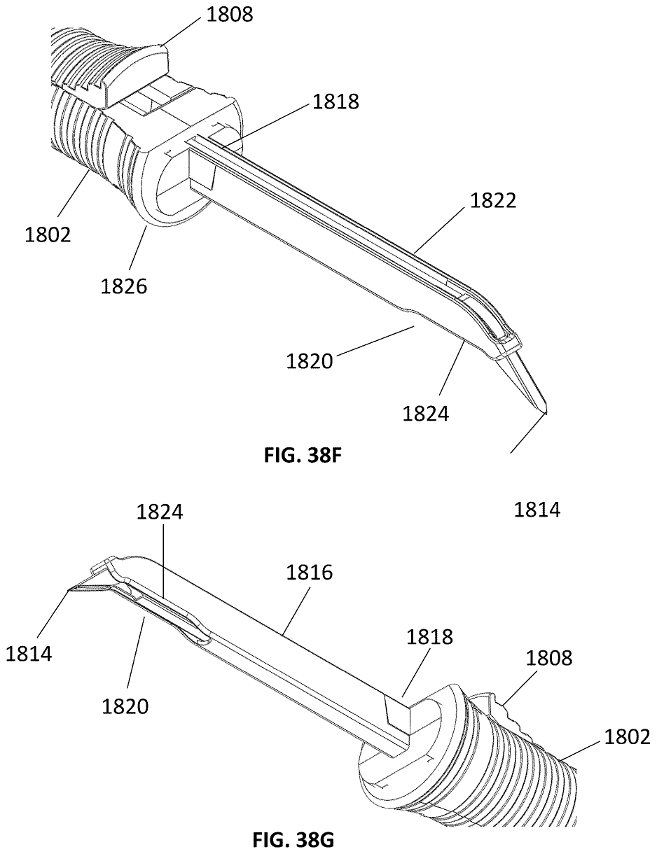

Referring to FIGS. 38A-38C, delivery system (1800) may include a handle (1802) and a cannula (1804) extending therefrom. The cannula (1804) may have a non-circular (e.g., rectangular) cross-sectional shape along a portion of its length, and a curved distal portion (1806). Additionally, the handle (1802) may include a lock (1808) configured to permit translational movement of an actuator (1810) when depressed. The actuator (1810) may advance a pusher (not shown) of the delivery system, which in turn advances a reshaping device coupled thereto from the cannula into tissue. Although shown as a depressible button, lock (1808) may have other configurations.

In some variations, as shown in FIG. 38D, the delivery system (1800) may include a tip protector (1812) for placement on the sharp distal tip (1814) of the cannula (1804) when the delivery system (1800) is not in use. For example, the tip protector (1812) may be placed on the sharp distal tip (1814) during packaging and removed prior to the start of a procedure. Additionally, as shown in FIGS. 38E-38G, some variations of the delivery systems described herein may include a depth guard (1816) having a proximal end (1818) and a distal end (1820) that may be configured for removable attachment to the cannula (1804). The depth guard (1816) may include a first cutout (1822) configured to run along the top of the depth guard (1816), and a second cutout (1824) configured to run along the distal bottom portion of the depth guard (1816). The first, top cutout (1822) may be longer than the second, bottom cutout (1824). The length, shape, and/or geometry of the first and second cutouts (1822, 1824) may be selected to allow for efficient removal and/or reattachment of the depth guard (1816) to the cannula (1804) and the distal end (1826) of the handle (1802). For example, as shown in the cross-sectional view of the delivery system (1800) in FIG. 38C, a clip (1828) configured to attach and/or detach the depth guard (1816) to a proximal tab (1830) of the handle (1802) and over the cannula may be provided at the distal end (1826) of the handle (1802).

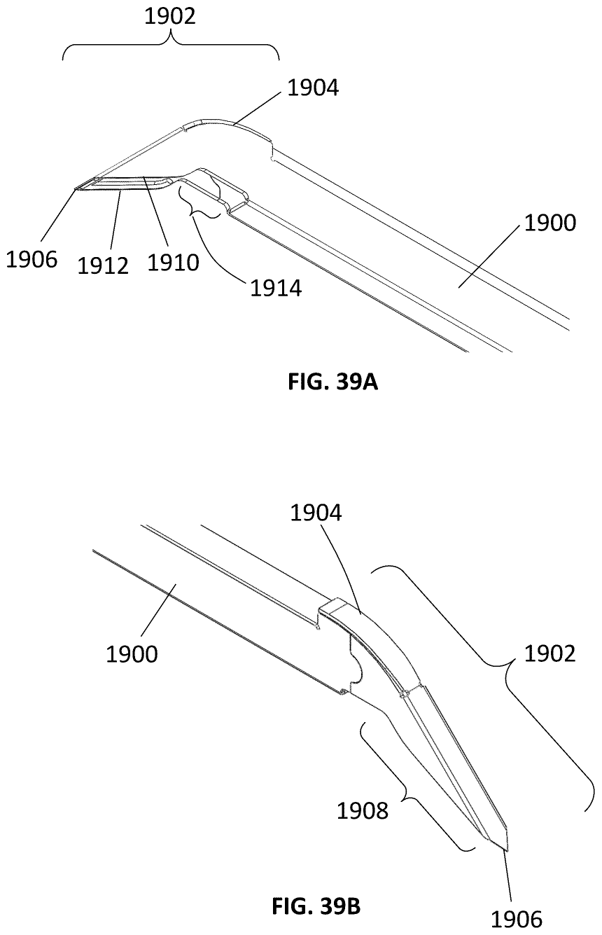

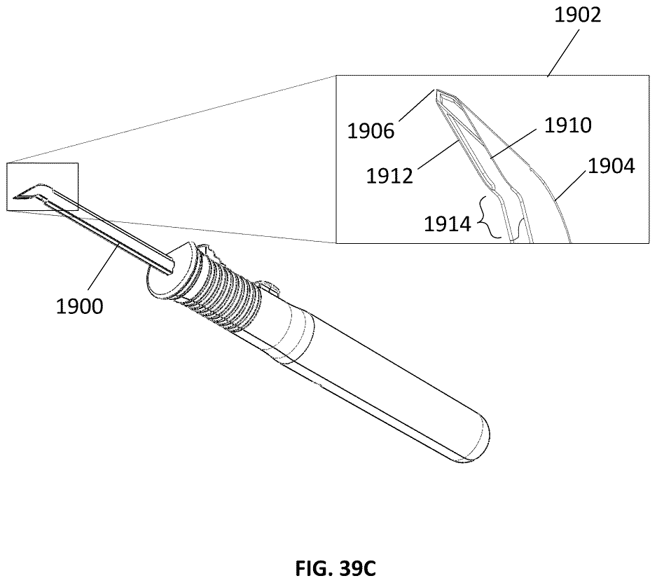

As previously mentioned, the cannula may have a distal portion comprising a curved upper surface, a sharp distal tip, and a lower surface comprising a first cutting edge and a second cutting edge, where the first and second cutting edges and the sharp distal tip are configured to create an arc-shaped incision in a target nasal tissue, e.g., the nasal septum. For example, referring to FIGS. 39A-39C, cannula (1900) may comprise a distal portion (1902) including a curved upper surface (1904), a sharp distal tip (1906), and a lower surface (1908) comprising a first cutting edge (1910) and a second cutting edge (1912). The first and second cutting edges (1910, 1912) and the sharp distal tip (1906) are generally configured to create an arc-shaped incision in a target nasal tissue, e.g., the nasal septum, as shown in FIGS. 37A and 37B. Additionally, the distal portion (1902) of the cannula (1900) may comprise a stop region (1914) configured to limit a puncture depth of the sharp distal tip (1906). The stop region (1914) may be a recessed and/or wider region of the distal portion (1902) of the cannula (1900). In some variations, the stop region (1914) may be a cutout in the distal portion (1902) of the cannula (1900).

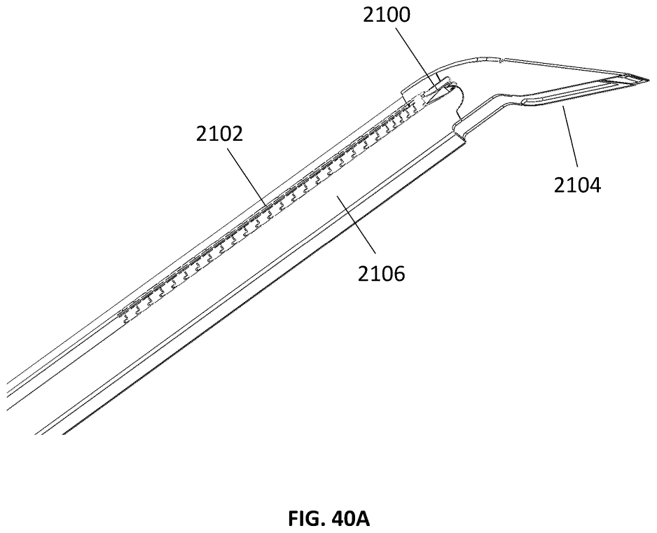

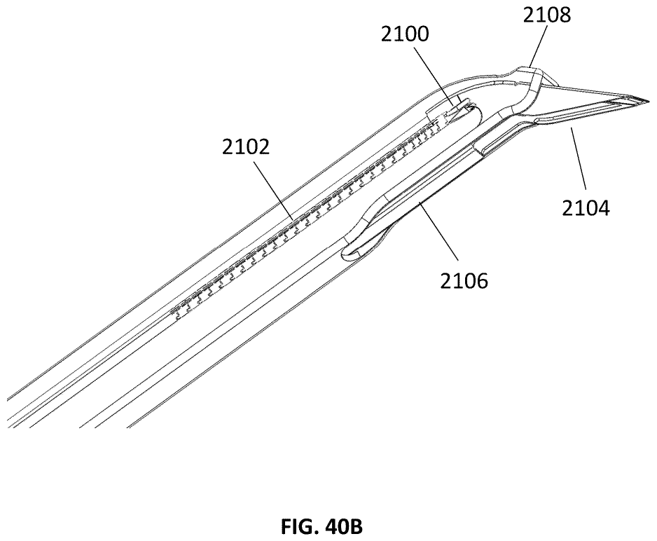

In use, a tension element may be deployed from the delivery systems via a pusher. Referring to FIG. 40A, the retainer of a tension element (e.g., retainer (1524) of tension element (1500) in FIG. 35) may be releasably attached to a coupler (2100) of the pusher (2102). Translation of the actuator (e.g., actuator (1810) in FIGS. 38A-38G) may then advance the tension element through a distal opening (2104) of the cannula (2106). A depth guard (2108) may be provided on the cannula (2106), as shown in FIG. 40B.

In some variations, the method for reshaping a nasal tissue of a subject may include creating an incision in the nasal tissue with a cannula; advancing a device through the cannula, the device comprising a tension element having an elongate body, a distal anchor, and a pivot point, where the distal anchor comprises a first arm and a second arm, and where the length of the first arm from the pivot point is different from the length of the second arm from the pivot point; advancing the distal anchor in an insertion configuration from the cannula through the nasal tissue; and applying a force to the tension element to rotate the distal anchor about the pivot point and transition the distal anchor to the deployed configuration. The incision may be U-shaped or arc-shaped. As previously stated, the reshaping device may be preloaded within the cannula. Advancement of the distal anchor and one or more proximal anchors through nasal tissue may be accomplished using a pusher. The nasal tissue may be nasal septal tissue, or other tissues in the nose. For example, the nasal tissue may comprise one or more of nasal septal cartilage, lateral nasal cartilage, major alar cartilage, minor alar cartilage, alar fibrofatty tissue, and nasal bone. The tension elements and delivery systems described herein may be used to treat nasal septal deviation, nasal valve collapse, and/or nasal airway obstruction.

In one variation, the method for reshaping nasal septal tissue to fix/treat a nasal septal deviation may include first creating a septoplasty incision (e.g., a Killian-like or hemitransfixion-like incision) in the nasal septal mucosa on the convex side of the deviation. A subperichondrial flap may then be made (e.g., using a freer elevator, Cottle elevator, or equivalent) to access the nasal septal cartilage. Next, the cannula of a delivery system, e.g., as shown in FIGS. 38A-38G and 39A-39C, may be advanced into the subperichondrial flap towards a target cartilage puncture site. The target site may be the deployment site of the distal anchor of the tension element, which may generally be anterior to the bony/cartilaginous junction. The cannula may then be advanced with the sharp tip directed parallel to the nasal septum to minimize the profile of the cannula beneath the flap and the cartilage incised by rotating the sharp tip of the cannula into the cartilage and applying medial force until the sharp tip punctures the cartilage. The cannula tip may then be advanced across the nasal septum until it is prevented from moving further by the stop region and/or depth guard. In some variations, the sharp distal tip may also puncture the contralateral septal mucosa in addition to the cartilage (although puncture of the mucosa may not be a critical step). The user may next depress the lock on the delivery system handle to unlock the actuator so that the actuator may slide forward (towards the cannula distal tip) to deploy the distal anchor of the tension element out of the cannula. In some instances, this step may be performed before advancing the cannula in the subperichondrial flap so that the actuator is ready to deploy the tension element immediately after the incision is made.

The actuator may advance a pusher including a coupler at its distal end attached to the distal anchor of a tension element, which in turn may advance the tension element out the distal end of the cannula and deploy the distal anchor on the contralateral side of the nasal septum. The delivery system may then be withdrawn back through the submucosal flap by retracting the handle straight back towards the user, leaving the distal anchor anchored on the contralateral side of the nasal septum. A needle driver (or other equivalent device) may be used to drive the proximal needle of the tension element through an anterior portion of the nasal septum. A force may then be applied to the tension element, which may generally span the apex of the convex side of the deviation, to reshape the nasal septum. After the desired amount of reshaping is achieved, the excess length of the tension element may be trimmed. The applied tension may generally be maintained on the nasal septal tissues by the plurality of proximal anchors of the tension element implanted within the nasal septal tissues.

### Manipulation of Nasal Turbinates

When nasal turbinate tissue is to be manipulated (reshaped, repositioned, compressed, approximated, etc.), the device may include an elongate body having a distal and a proximal end, a distal anchor at the distal end comprising at least a first arm and a second arm (e.g., a T-anchor), and a plurality of proximal anchors disposed along a length of the elongate body between the distal anchor and the proximal end, where the size of the plurality of proximal anchors decreases along the length of the elongate body from the distal anchor towards the proximal end. Stated differently, the tension element may include a plurality of proximal anchors having a decreasing size gradient along a region/length of the elongate body from the distal anchor towards the proximal end.

The plurality of proximal anchors may comprise one or more of a barb, hook, spike, tooth, leaf, nub, and protrusion. A distal end of each of the plurality of proximal anchors may be beveled, tapered, sharp, curved, or a combination thereof. In some variations, the plurality of proximal anchors may be symmetrically spaced apart along a length of the elongate body. In other variations, the plurality of proximal anchors may be asymmetrically spaced apart along a length of the elongate body. The plurality of proximal anchors may be staggered (offset) along the length of the elongate body. In some variations, a first set of the plurality of proximal anchors may be offset along a longitudinal axis of the elongate body from a second set of the plurality of proximal anchors. The plurality of proximal anchors may be provided on a region of the elongate body having a length ranging from about 0.5 cm to about 3.5 cm, including all values and ranges therein. For example, the plurality of proximal anchors may be provided on a portion of the elongate body having a length of about 0.5 cm, about 1.0 cm, about 1.5 cm, about 2.0 cm, about 2.5 cm, about 3.0 cm, or about 3.5 cm. In one variation, the plurality of proximal anchors may be provided on a portion of the elongate body having a length of about 1.0 cm.

The distance (e.g., spacer length) between the distal anchor and the first proximal anchor (e.g., the most distal anchor of the plurality of proximal anchors) of the plurality of proximal anchors may range from about 4.0 mm to about 6.0 mm, including all values and sub-ranges therein. For example, the distance between the distal anchor and the first proximal anchor of the plurality of proximal anchors may be about 4.0 mm, about 4.1 mm, about 4.2 mm, about 4.3 mm, about 4.4 mm, about 4.5 mm, about 4.6 mm, about 4.7 mm, about 4.8 mm, about 4.9 mm, about 5.0 mm, about 5.1 mm, about 5.2 mm, about 5.3 mm, about 5.4 mm, about 5.5 mm, about 5.6 mm, about 5.7 mm, about 5.8 mm, about 5.9 mm, or about 6.0 mm. In some variations, the first proximal anchor of the plurality of proximal anchors may be spaced from the distal anchor a distance of about 4.8 mm or about 4.9 mm. When the middle turbinates are to be medialized towards the nasal septum, the spacer length may be selected such that none of the plurality of proximal anchors contacts the nasal septum after tensioning and securement of the elongate body. In this instance, the spacer length may be between about 4.0 mm to about 5.0 mm, including all values and sub-ranges therein.

As described above, the elongate body of the tension element may be flexible, and may be straight or have at least a portion with an undulating or wave-like shape. In some variations, at least the portion of the elongate body upon which the proximal anchors are disposed has an undulating or wave-like shape. A needle may be releasably coupled to the proximal end of the elongate body.

In some variations, the tension element for manipulating nasal turbinates may comprise a biodegradable polymer. The biodegradable polymer may be selected from the group consisting of LPLA (Poly(L-lactide)), DLPLA (Poly(DL-lactide)), LDLPLA (Poly(DL-lactide-co-L-lactide)), LPLA-HA (Poly(L-lactide) with hydroxylapatite), PGA (Poly(glycolide)), PGA-TMC (Poly(glycolide-co-trimethylene carbonate) or polyglyconate), PDO (Poly(dioxanone)), LPLG (Poly(L-lactide-co-glycolide)), DLPLG (Poly(DL-lactide-co-glycolide), and copolymers and blends thereof. In one variation, the tension element may comprise PDO (Poly(dioxanone)). In other variations, the tension element may comprise a non-biodegradable polymer or metal, as previously described herein.

An exemplary device for manipulating nasal turbinates is shown in FIG. 36. Referring to FIG. 36, the tension element (1600) may include an elongate body (1602) having a proximal end (1604), a distal end (1606), and a distal anchor (1608) at the distal end (1606). The distal anchor (1608) may include a first arm (1610) and a second arm (1612). Although the distal anchor (1608) in FIG. 36 is shown as a T-anchor with arms of equal length, it is understood that their lengths may be different. The T-anchor configuration may allow the distal anchor to sit flat against a nasal turbinate and thus minimize the collection of mucus or formation of crusts in the area. Furthermore, the tension element (1600) may include a plurality of proximal anchors (1614). The plurality of proximal anchors (1614) may comprise leaves (1616, “leaf anchors”) that are staggered/offset from each other along a region/length of the elongate body (1602). The length of the elongate body along which the proximal anchors (1614) are disposed may have an undulating or wave-like shape and be about 1.0 cm. The plurality of proximal anchors (1614) may be spaced about 1.5 mm from each other. In addition, as previously mentioned, a first proximal anchor (1618) may be spaced from the distal anchor (1608) a distance between about 2.0 mm to about 6.0 mm, including all values and sub-ranges therein. The free ends (distal tips) of the plurality of proximal anchors may extend distally towards the distal anchor. When used for medialization of both middle turbinates, it may be useful for the first proximal anchor (1618) of the tension element (1602) to be spaced from distal anchor (1608) about 4.9 mm. This amount of spacing may prevent any proximal anchor from resting on the nasal septum after the tension element has been tensioned and secured in place.

In addition to being staggered/offset, the plurality of proximal anchors (1614) may have a decreasing size gradient along the region/length of the elongate body from the distal anchor (1608) towards the proximal end (1604). Stated differently, a first proximal anchor (1618) of the plurality of proximal anchors (1614) may be larger than a last proximal anchor (1620) of the plurality of proximal anchors (1614), with the proximal anchors therebetween gradually decreasing in size from the first proximal anchor (1620) to the last proximal anchor (1620). For example, the first proximal anchor (1618) may have a length of about 2.25 mm, and the last proximal anchor (1620) may have a length of about 0.25 mm, with the length of the proximal anchors therebetween gradually decreasing from about 2.25 mm to about 0.25 mm. In some variations, the first proximal anchor (1618) may have a length of about 1.75 mm, and the last proximal anchor (1620) may have a length of about 0.75 mm, with the length of the proximal anchors therebetween gradually decreasing from about 1.75 mm to about 0.75 mm. The decreasing size gradient may allow a larger anchor to reside within the turbinate and the tension element to be cut flush with mucosa of the turbinate. A needle (1622) may be releasably coupled to the proximal end (1604) of the tension element (1602).

When both middle turbinates are to be medialized, the proximal needle (1622) may be inserted through a first middle turbinate, the nasal septum, and then a second middle turbinate, e.g., using a needle driver (or other equivalent device). Applying a force to the tension element (1602) may then pull the first middle turbinate medially toward the nasal septal cartilage and secure the distal anchor (1608) against the first middle turbinate, as similarly described above for FIGS. 26A and 26B. The medial position of the second middle turbinate may be maintained by securing one or more proximal anchors (1616), and generally a larger proximal anchor of the plurality of proximal anchors (1614) within the second middle turbinate. In some variations, the method for manipulating nasal turbinates in a subject may include advancing a tension element through a first nasal turbinate on a first side of a nasal septum, the tension element comprising an elongate body having a proximal end and a distal end, a distal anchor at the distal end comprising at least a first arm and a second arm, and a plurality of proximal anchors disposed along a length of the elongate body between the distal anchor and the proximal end, where a size of the plurality of proximal anchors decreases along the length from the distal anchor towards the proximal end; advancing the tension element through the nasal septum; advancing the tension element through a second nasal turbinate on a second side of the nasal septum opposite the first side; and applying a force to the tension element to position the distal anchor against an outer surface of the first nasal turbinate and one or more of the plurality of proximal anchors within the second turbinate. The method may further include securing one or more of the plurality of proximal anchors within the second nasal turbinate. These proximal anchors may be larger anchors within the size gradient of proximal anchors. After medialization is accomplished, the tension element may be cut flush with an outer surface of the second nasal turbinate. In one variation, the first and second nasal turbinates are middle turbinates. In some variations, other nasal turbinates (e.g., inferior turbinates, supreme turbinates) may be medialized or lateralized.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated.

1. A device for manipulating one or more nasal turbinates comprising:
a tension element, the tension element comprising an elongate body having a proximal end and a distal end;
a distal anchor at the distal end comprising at least a first arm and a second arm; and
a plurality of proximal anchors disposed along a region of the elongate body between the distal anchor and the proximal end, wherein a length of the plurality of proximal anchors decreases along the region of the elongate body from the distal anchor towards the proximal end.

2. The device of claim 1, wherein the distal anchor comprises a T-anchor.

3. The device of claim 1, wherein the plurality of proximal anchors comprises one or more of a barb, hook, spike, leaf, nub, or protrusion.

4. The device of claim 1, wherein a distal end of each of the plurality of proximal anchors is beveled, tapered, sharp, curved, or a combination thereof.

5. The device of claim 1, wherein a first proximal anchor of the plurality of proximal anchors is spaced between about 2.0 mm to about 6.0 mm from the distal anchor.

6. The device of claim 5, wherein the first proximal anchor of the plurality of proximal anchors is spaced about 5.0 mm from the distal anchor.

7. The device of claim 1, wherein a first set of the plurality of proximal anchors are offset along a longitudinal axis of the elongate body from a second set of the plurality of proximal anchors.

8. The device of claim 1, wherein the plurality of proximal anchors is symmetrically spaced apart along a length of the elongate body.

9. The device of claim 1, wherein the plurality of proximal anchors is asymmetrically spaced apart along a length of the elongate body.

10. The device of claim 1, wherein the region of the elongate body upon which the plurality of proximal anchors is disposed has an undulating shape.

11. The device of claim 1, further comprising a needle releasably coupled to the proximal end of the elongate body.

12. The device of claim 1, wherein the tension element comprises a biodegradable polymer.

13. The device of claim 12, wherein the biodegradable polymer is selected from the group consisting of LPLA (Poly(L-lactide)), DLPLA (Poly(DL-lactide)), LDLPLA (Poly(DL-lactide-co-L-lactide)), LPLA-HA (Poly(L-lactide) with hydroxylapatite), PGA (Poly(glycolide)), PGA-TMC (Poly(glycolide-co-trimethylene carbonate) or polyglyconate), PDO (Poly(dioxanone)), LPLG (Poly(L-lactide-co-glycolide)), DLPLG (Poly(DL-lactide-co-glycolide), and copolymers and blends thereof.

14. The device of claim 12, wherein the biodegradable polymer comprises PDO (Poly(dioxanone)).

15. The device of claim 1, wherein the one or more nasal turbinates comprises a middle turbinate, an inferior turbinate, a superior turbinate, or a supreme turbinate.

16. A method for manipulating nasal turbinates in a subject comprising:
advancing the tension element of claim 1 through a first nasal turbinate on a first side of a nasal septum;
advancing the tension element through the nasal septum;
advancing the tension element through a second nasal turbinate on a second side of the nasal septum opposite the first side; and
applying a force to the tension element to position the distal anchor against an outer surface of the first nasal turbinate and one or more of the plurality of proximal anchors within the second turbinate.

17. The method of claim 16, further comprising securing one or more of the plurality of proximal anchors within the second nasal turbinate.

18. The method of claim 17, further comprising cutting the tension element flush with an outer surface of the second nasal turbinate.

19. The method of claim 16, wherein the first and second nasal turbinates are middle turbinates.

20. The method of claim 19, wherein applying the force to the tension element medializes the first and second middle turbinates towards the nasal septum.

21. The method of claim 16, wherein applying the force to the tension element lateralizes one or more of the first or second nasal turbinates.
